(19) United States
(12) Patent Application Publication
(10) Pub. No.: US 2025/0019658 A1
(43) Pub. Date: Jan. 16, 2025

(54) MATURATION MEDIUM FOR PLURIPOTENT STEM CELL-DERIVED CARDIOMYOCYTES

(71) Applicant: THE GOVERNING COUNCIL OF THE UNIVERSITY OF TORONTO, Toronto (CA)

(72) Inventors: Neal I. CALLAGHAN, Brooklyn (CA); Craig A. SIMMONS, Toronto (CA)

(21) Appl. No.: 18/711,292

(22) PCT Filed: Nov. 17, 2022
(86) PCT No.: PCT/CA2022/051700

§ 371 Date: May 17, 2024

Related U.S. Application Data
(60) Provisional application No. 63/280,388, filed on Nov. 17, 2021.

(51) Int. Cl. C12N 5/077

(52) U.S. Cl. CPC C12N 5/0657; C12N 2500/25; C12N 2500/33; C12N 2500/34; C12N 2500/36; C12N 2500/46; C12N 2501/105; C12N 2501/235; C12N 2501/39

(57) ABSTRACT

A culture medium is provided for the maturation of pluripotent stem-cell derived cardiomyocytes. The culture medium contains a number of soluble factors, which can include putrescine, leukemia inhibitor factor and metformin.

### TECHNICAL FIELD

This invention relates to cell culture medium compositions and compositions and methods for use in the maturation of immature cardiomyocytes.

### BACKGROUND OF THE ART

Induced pluripotent stem cells (iPSCs) and iPSC-derived cardiomyocytes (iPSC-CMs) offer tremendous promise for basic cardiovascular science, disease modeling, drug efficacy and toxicity testing, personalized medicine and cell therapy to restore myocardial function during heart failure or post-infarction. However, current iPSC-CMs are physiologically immature in all categories of relevant function, including morphology, contractility, calcium handling, action potential electrophysiology, and metabolism, which is a major limitation towards the application and commercialization of iPSC-CMs.

### BRIEF SUMMARY

In one aspect, there is provided an aqueous cell culture medium that includes: a high-flux carbon source or central carbon metabolism inhibitor; at least one secondary metabolite; and at least one of putrescine, leukemia inhibitory factor (LIF) and metformin. In one embodiment, the cell culture medium includes about 0.001 mM to about 0.03 mM putrescine, about 0.1 ng/mL to about 10 ng/mL LIF and/or about 0.001 mM to about 0.25 mM metformin.

The high-flux carbon source or central carbon metabolism inhibitor may be a lipid mixture, lactate, galactose or a combination of any of the foregoing. In some embodiments, the lipid mixture comprises cholesterol and one or more lipids selected from: linoleic acid, linolenic acid, myristic acid, oleic acid, palmitic acid, palmitoleic acid and stearic acid.

The at least one secondary metabolite may be creatinine, carnitine, taurine, ethanolamine or a combination of any of the foregoing. In some embodiments, the culture medium includes about 0.001 mM to about 6.5 mM creatinine, about 0.001 mM to about 6.5 mM carnitine, about 0.001 mM to about 6.5 mM taurine, about 0.001 mM to 0.03 mM putrescine, and about 0.001 mM to about 0.01 mM ethanolamine.

The cell culture medium may further include one or more hormones selected from the group consisting of triiodothyronine (T3), insulin-like growth factor (IGF), hydrocortisone, human growth hormone, neuregulin and insulin, preferably T3, IGF, hydrocortisone, neuregulin and insulin. In some embodiments, the culture medium includes about 0.001 ng/mL to 80 ng/mL T3, about 0.001 ng/mL to about 100 ng/mL IGF, about 0.001 ng/mL to about 80 ng/mL hydrocortisone about 0.001 ng/mL to about 5 ng/mL neuregulin and about 0.75 g/L to about 1.25 g/L insulin.

The cell culture medium suitably further includes transferrin and selenium.

The cell culture medium suitably further includes an antioxidant, preferably a synthetic antioxidant, preferably β-mercaptoethanol.

In one embodiment, the cell culture medium includes:

| | |
|---|---|
| 0 mM-0.03 mM | Putrescine |
| 0 mM-0.25 mM | Metformin |
| 0 ng/mL-10 ng/mL | Leukemia inhibitory factor |

| | | |
|---|---|---|
| 0-10 | mM | Lactate |
| 0-3 | g/L | A lipid mixture |

| | |
|---|---|
| 0 mM-40 mM | Galactose |
| 0 mM-6.5 mM | Creatine |
| 0 mM-6.5 mM | Carnitine |
| 0 mM-6.5 mM | Taurine |

| | | |
|---|---|---|
| 0-0.3 | mM | Ethanolamine |
| 0-1.25 | g/L | Insulin |
| 0-0.7 | g/L | Transferrin |
| 0-1 | mg/L | Selenium |
| 1-3 | wt % | Albumin |
| 0-0.1 | mM | β-mercaptoethanol |

| | |
|---|---|
| 0 ng/mL and 80 ng/mL | Triiodothyronine |
| 0 ng/mL and 100 ng/mL | Insulin-like growth factor |
| 0 ng/mL and 100 ng/mL | Human growth hormone |
| 0 ng/mL and 80 ng/mL | Hydrocortisone |
| 0 ng/mL and 5 ng/mL | Neuregulin |

In other embodiments, the cell culture medium consists or consists essentially of the listed components, and, in some embodiments, optionally a commercially available chemically defined medium, such as Medium 199, one or more antibiotics and/or saline solution.

In one embodiment, the cell culture medium includes:

| | |
|---|---|
| 0.02 mM-0.025 mM | Putrescine |
| 0.05 mM-0.10 mM | Metformin |

| | | |
|---|---|---|
| 2-8 | ng/mL | Leukemia inhibitory factor |
| 0.1-3 | g/L | A lipid mixture |

| | |
|---|---|
| 15 mM-25 mM | Galactose |
| 0.1 mM-1 mM | Creatine |
| 0.1 mM-1 mM | Carnitine |
| 0.1 mM-1 mM | Taurine |
| 0.001 mM-0.01 mM | Ethanolamine |

| | | |
|---|---|---|
| 0.1-1.25 | g/L | Insulin |
| 0.1-0.7 | g/L | Transferrin |
| 0.1-1 | mg/L | Selenium |
| 1-3 | wt % | Albumin |

| | |
|---|---|
| 0.001 mM-0.1 mM | β-mercaptoethanol |
| 1 ng/mL-10 ng/mL | Triiodothyronine |
| 10 ng/mL-40 ng/mL | Insulin-like growth factor |
| 1 ng/mL-10 ng/mL | Hydrocortisone |
| .01 ng/mL-1 ng/mL | Neuregulin |

In other embodiments, the cell culture medium consists or consists essentially of the listed components, and, in some embodiments, optionally a commercially available chemically defined medium, such as Medium 199, one or more antibiotics and/or saline solution.

In some embodiments, the high-flux carbon source may include 0.001 mM to 20 mM 2-deoxyglucose, preferably 5 mM to 10 mM 2-deoxyglucose. In other embodiments, the cell culture medium is substantially free of 2-deoxyglucose.

The culture medium is suitably a serum-free medium.

The cell culture medium may further include Media 199.

The cell culture medium may further include at least one antibiotic, preferably penicillin and/or streptomycin.

Also provided is a kit that includes a culture medium as provided herein and immature cardiomyocytes or undifferentiated pluripotent embryonic stem cells (PESC) and/or induced pluripotent stem cells (iPSC) and a differentiation medium for differentiating PESC and/or iPSC into cardiomyocytes.

Also provided is a composition for improving the cardiomyocyte maturing properties of a cell culture medium, the composition including one or more of putrescine, leukemia inhibitory factor (LIF) and metformin, in one embodiment any two of putrescine, LIF and metformin and in one embodiment all of putrescine, LIF and metformin. The composition may be a solid or semi-solid that can be dissolved in an aqueous solution so as to obtain a cell culture medium as provided herein.

Also provided is a method of maturing immature cardiomyocytes to obtain adult-like cardiomyocytes by contacting immature cardiomyocytes with a culture medium as provided herein for a time sufficient to effect maturation of the immature cardiomyocytes. The method can further include differentiating PESC and/or iPSC to obtain the immature cardiomyocytes. The immature cardiomyocytes may be contacted with the culture medium for a period of 10-45 days, in one embodiment for a period of 14-21 days. In one embodiment, the cells are cultured at a concentration of 50K to 250K cells per cm².

The method may include co-culturing the immature cardiomyocytes with non-cardiomyocyte cells, preferably epicardial cells and/or cardiac fibroblasts. The immature cardiomyocytes may be contacted with the culture medium in a 3D tissue culture system, preferably an engineered tissue or organoid.

Also provided are adult-like cardiomyocytes matured by a method provided herein and a composition comprising such adult-like cardiomyocytes and a pharmaceutically acceptable carrier or biocompatible scaffold. Also, provided herein are microtissues comprising adult-like cardiomyocytes matured by methods provided herein. In some embodiments, the adult-like cardiomyocytes and/or microtissues as provided herein exhibit minimal spontaneous contractility, as compared to adult-like cardiomyocytes and/or microtissues generated therefrom matured in prior cell culture mediums, including those identified herein.

### BRIEF DESCRIPTION OF THE DRAWINGS

FIG. 1. Iterative development and performance of iPSC-CM maturation medium candidates relative to a commercial gold-standard formulation. a) Visual representation of high dimensional, directed evolution (HD-DE) algorithm workflow; from a quasi-random distribution of vectors (formulations) in an extensive but discrete solution space created by integer doses of multiple additive factors, low-performing candidates are discontinued while high-performing candidates are kept and iterated using mutation and crossover operations. b) Ranked performance of all tested formulation candidates (N=169 unique vectors over 204 instances) against STEMCELL Technologies Cardiomyocyte Maintenance Medium (dashed line), using respirometric uncoupled-control ratio as an objective metric. c) Principal component analysis biplot visualization of the top ten global performing formulations in addition to a composite, manually-defined formulation (grey points indicate 1^{st}, 2^{nd}, 3^{rd}, and Custom compositions as labeled); grey vector arrows represent correlation of medium additives. d) Relief contrast micrographs post 3 weeks culture in global top-two performing formulations, the Custom formulation chosen to test in detail, or STEMCELL Cardiomyocyte Maintenance Medium. Scale bars 200 μm. e) Relative displacement traces from particle-image velocimetry of monolayers cultured 6 weeks in Custom or STEMCELL media. (f) Normalized displacement curves of spontaneously-contracting Custom- or RPMI+B27-treated iPSC-CM monolayers (individual traces pale, n=4 each treatment; quartic fits dark) derived from PGPC17, PGPC14, and PGPC17-MYBPC3-KO iPSC lines. All analyses performed after 3 weeks of treatment with medium formulations.

FIG. 2. Morphology of iPSC-CMs is enhanced by 6 weeks of culture in Custom PSC-CM maturation medium. Confocal images of a) immunodetected α-actinin (top) and Cx43 (middle), or DAPI-stained nuclei (bottom), or b) phalloidin-stained F-actin (top) and DAPI-stained nuclei (bottom) of monolayers cultured in Custom, iCell, RPMI+B27, or STEMCELL media; DAPI-stained nuclei. Channel contrast is maintained between treatments for each imaging condition; scale bars 50 μm.

FIG. 3. Metrics of Ca²⁺ handling are enhanced using Custom PSC-CM maturation medium. a) Representative line-scanning Fluo-4 traces during steady-state Ca²⁺ transients at 1 Hz pacing of PSC-CMs cultured in Custom formulation and gold-standard iPSC-CM media (iCell Cardiomyocytes Maintenance Medium, RPMI+B27, and STEMCELL Cardiomyocyte Maintenance Medium), and b) their respective kinetic metrics (time to peak, and times to 10%, 25%, 50%, and 75% decay respectively). c) Change in kinetic metrics before and after addition of 5 μmol L⁻¹ isoproterenol. Fluo-4-enabled Ca²⁺ traces of Custom (d) and STEMCELL (e)-treated cultures after 6 weeks; with steady-state pacing at 1 Hz followed by sequential additions of verapamil (20 μmol L⁻¹) and caffeine (10 mmol L⁻¹). f Caffeine-induced Ca²⁺ unloading amplitudes normalized to steady state transient amplitudes; each data point represents an individual cell (N=3 biological replicates of Custom, 2 biological replicates of STEMCELL). Significance indicated by *p<0.05, **p<0.01, ***p<0.001, and ****p<0.0001 by Welch's t-test/Dunnett's post-hoc test; error bars denote mean±SD.

FIG. 4. Oxidative metabolism of iPSC-CMs is enhanced by culture in Custom PSC-CM maturation medium. Representative oxygen consumption rate (a) and extracellular acidification rate (b) traces of mitochondrial stress test of iPSC-CMs cultured 6 weeks in Custom medium or existing gold-standard control formulations, normalized to cell number. c) Basal oxygen consumption rates and d) uncoupled-control ratios by treatment. Significance indicated by *p<0.05 by one-way ANOVA/Tukey's HSD; error bars denote mean±SD.

FIG. 5. Global proteomic profiling of Custom-treated iPSC-CMs relative to gold-standard formulations. Principal component analysis of differential protein expression in cells after 6 weeks of treatment in Custom, iCell, RPMI+B27, STEMCELL media, as well as a top-performing formulation from the literature [Filice D, Dhahri W, Solan J L, et al (2020) Optical mapping of human embryonic stem cell-derived cardiomyocyte graft electrical activity in injured hearts. Stem Cell Res Ther 11:417].

FIG. 6. Transcriptomic profiling of Custom-treated iPSC-CMs. a) Principal component analysis of iPSC-CMs cultured in Custom medium or a gold-standard formulation for 6 weeks, or at d20 of differentiation and prior to treatment (control). b) Heatmaps displaying expression of select genes from the GO terms: ‘oxidative phosphorylation’ (top), ‘cardiac muscle action potential’ (middle) and ‘heart contraction’ (bottom). c) Mean normalized counts of select genes associated with CM maturation. N=3 biological replicates; error bars denote mean±SEM. Significance indicated by *p<0.05, **p<0.01, and ***p<0.001 by one-way ANOVA/Tukey's HSD.

FIG. 7. Transcriptionally-enriched functional terms and pathways in Custom-treated iPSC-CMs. a) Top 20 enriched GO Biological Process terms among significantly upregulated genes in the indicated comparisons. Point size denotes the size of the enriched gene set corresponding to each term. b) Top 20 enriched KEGG pathways in the indicated comparisons. Point size denotes the size of the enriched gene set corresponding to each pathway.

FIG. 8. Electrophysiological enhancement of iPSC-CMs matured using Custom medium. a) Sample traces of intracellular electrode recordings of iPSC-CMs treated with Custom maturation medium (left) or RPMI+B27 (right) for 6 weeks, in neat Tyrode's solution, followed by sequential supplementation with 100 and 400 μmol L-1 BaCl₂, before eventual washout. b) Minimum diastolic potentials (resting membrane potentials) from intracellular recording in cell sheets, with or without 400 μmol L-1 BaCl₂ perfusion. c) Spontaneous action potential firing rates measured during action potential recordings, with or without 400 μmol L-1 BaCl₂ perfusion. d) Inward rectifier (IK1) currents in neat Tyrode's and supplemented with 400 μmol L-1 BaCl₂; inset indicates voltage protocol and scale. e) Voltage sensitivity curves of IK1; inset demonstrates positive component of IK1 at high voltage. D IK1 current density at −115 mV. Data points denote biological replicates. Error bars denote mean±SD *, **, ***, and *** denote p<0.05, 0.01, 0.001 and 0.0001, respectively.

FIG. 9. Contractile enhancement of engineered myocardial tissues cultured in Custom minus 2DG maturation medium. a) Decrease in tissue contractile spontaneity after 3 weeks of culture in Custom minus 2DG media. b) Progressive absolute twitch force increase over 3 weeks of Biowire II culture in Custom minus 2DG or RPMI+B27 medium formulations, with or without continuous electrical field stimulation (1 Hz). c) Positive force-frequency relationship of contractile displacement of polymeric beams summatively increases with both medium- and electrical stimulation-mediated maturation of tissues after 3 weeks of tissue treatment in Custom minus 2DG or RPMI+B27 media; measurements are normalized to 1 Hz stimulation of the relevant treatment condition (dotted line). d) Confocal microscopy reveals differential expression of SERCA2a (magenta) and DHPR (yellow) in electrically-stimulated Biowires cultured 3 weeks in Custom minus 2DG or RPMI+B27 media. e) TEM of longitudinal and transverse sections of Custom minus 2DG-treated Biowires induces sarcomeric development and higher ribosome-associated sarcoplasmic reticulum (scale bars 1 μm); detailed examination of Custom minus 2DG-treated tissues (e-h; scale bars 500 nm) include sarcomeres with defined M- and H-bands and Z-discs and myofibril-associated high mitochondrial density (f), electron-dense intercalated discs (g), and nuclear SR invaginations (h).

### DETAILED DESCRIPTION

### Definitions

As used herein, the term “cardiomyocytes” or “cardiac myocytes” (abbreviated herein as CMs) refers generally to any cardiomyocyte lineage cells, and can be taken to apply to cells at any stage of cardiomyocyte ontogeny, unless otherwise specified. For example, cardiomyocytes may include both cardiomyocyte precursor cells (immature cardiomyocytes or fetal cardiomyocytes) and mature cardiomyocytes (adult-like cardiomyocytes).

As used herein, “immature cardiomyocytes” refers to a cardiomyocyte derived from pluripotent embryonic stem cells (PESC) and/or induced pluripotent stem cells (iPSC) in in vitro culture, which do not possess the properties of an adult or adult-like cardiomyocyte.

As used herein, the term “induced pluripotent stem cells” (iPSC), refers to somatic (adult) cells reprogrammed to enter an embryonic stem cell-like state by being forced to express factors important for maintaining the “stemness” of embryonic stem cells. iPSC may be artificially prepared from a non-pluripotent cell, (i.e. adult somatic cell, or terminally differentiated cell) by introducing into or otherwise contacting the cell with reprogramming factors using techniques known to those of skill in the art.

As used herein, “pluripotent embryonic stem cells” (PESC) refers to pluripotent stem cells derived from the inner cell mass of a blastocyst. PESC can differentiate into cells derived from any of the three germ layers.

As used herein, “mature cardiomyocytes” or “adult-like cardiomyocytes” refers to cardiomyocytes which possess at least one, in some embodiments a number, and in some embodiments all, of the functional, transcript expression or protein expression associated with mature cardiomyocytes. Functional attributes associated with mature cardiomyocytes include contractility, electrophysiology, Ca2+ handling, metabolism and/or morphology.

As used herein, the term “differentiation” refers to a biological process whereby an unspecialized PESC or iPSC acquires the features of a specialized cell such as a cardiomyocyte under controlled conditions in in vitro culture via changes in gene expression. Methods of differentiating PESC or iPSC to cardiomyocytes and expression characteristics and morphological characteristics indicative of differentiation are available in the literature, e.g. in Lian et al (2013) Directed cardiomyocyte differentiation from human pluripotent stem cells by modulating Wnt β-catenin signaling under fully defined conditions. Nature Protocols 8:162-175; and Burridge et al. (2014) Chemically defined generation of human cardiomyocytes. Nature Methods 11: 855-860; the contents of which are incorporated herein in their entirety. Kits for differentiating stem cells are also commercially available, such as the Cardiomyocyte Differentiation Kit (STEMCELL Technologies).

Differentiation is controlled by the interaction of a cell's genes with the physical and chemical conditions outside the cell, usually through signalling pathways involving proteins embedded in the cell surface. In certain embodiments, pluripotent stem cells (e.g. PESC or iPSC) can be exposed to a cardiomyocyte differentiation medium so as to promote differentiation of pluripotent stem cells into immature, fetal-like cardiomyocytes. Cardiac differentiation can be detected by the use of markers selected from, but not limited to, NKX2-5, GATA4, myosin heavy chain, myosin light chain, alpha-actinin, troponin, and tropomyosin (Burridge et al (2012) Stem Cell Cell, Vol. 10(1):16-28, US2013/0029368).

As used herein, “maturing” and grammatical variations thereof refers to a process that renders one or more of the functional, transcript expression or protein expression of immature cardiomyocytes more adult-like i.e. renders one or more of these functional, transcript expression or protein expression more similar to that of mature adult-like cardiomyocytes.

### Indicia of Cardiomyocyte Maturation

CMs can be physiologically characterized on a functional basis, on a transcriptional basis, or on an expressional basis; said characterizations, either singly or as part of a larger complement of assays, can distinguish PSC-CMs matured by methods provided herein from mature in vivo CMs, as well as from PSC-CMs at either an unmatured state or after maturation using known culture mediums and methods. Characteristics of CMs as identified by such methods can be found in the literature, e.g. in Karbassi et al. (2020), Cardiomyocyte maturation: advances in knowledge and implications for regenerative medicine. Nat Rev Cardiol 17: 341-359, in particular at ¶0011.

### Functional:

Contractility: cells can be assessed (either as single cells, monolayers, clumps, or engineered constructs) by contractility (encompassing force production or contractile kinetics) using electrical impedance sensing of single cells or monolayers, visual methods (e.g. speckle tracking, traction force microscopy, sarcomeric diffraction, construct beam or cantilever bending), atomic force microscopy, or force transducer sensing. Increased peak twitch strain normalized per cross-sectional unit area is indicative of advancing physiological maturity, as are onset kinetics. Aspects of twitch force profiles (magnitude or kinetics) can increase in sensitivity to established positively or negatively inotropic agents based on the target cell's maturity.

Electrophysiology: cells can be assessed (either as single cells, monolayers, clumps, or engineered constructs) by intracellular or patch-clamp recordings for composite action potential waveforms, membrane potential, or the component currents of the cell, by using voltage-clamp, current-clamp, and a set of specific channel inhibitors; aggregate waveforms vary throughout the maturation process based on changing densities of specific composite currents.

Ca²⁺ handling: cells can be assessed (either as single cells, monolayers, clumps, or engineered constructs) by electrophysiology as described above, or more commonly by Ca²⁺ sensitive intracellular dyes such as Fluo-4 or the ratiometric dye Fura-2, among others, as measured using epifluorescent or confocal microscopy. Based on the degree of magnitude and resolution, Ca²⁺ events can be assessed either as aggregate transients, or on the level of individual Ca²⁺ sparks (clustered sarcoplasmic reticulum release bursts). Cells may differ in magnitude, kinetics, and sensitivity to pharmaceutical manipulation based on their maturity.

Metabolism: the metabolic state of PSC-CMs or native cardiomyocytes can be assessed in aggregate (as monolayers, clumps, or engineered constructs) by respirometry, biochemical assays, or targeted or untargeted metabolomic assays via mass spectroscopy. CM maturity is associated with an increase in oxidative phosphorylation flux, and a correspondingly increased preference for obligately oxidative substrates such as lactate, ketones, or fatty acids over anaerobic metabolism and glucose, respectively.

Morphology: matured cardiomyocytes exhibit high mitochondrial content, organization, and networking; maturity is also associated with high CM-specific cytoskeletal content, bundling and striation, and high transverse tubular and sarcoplasmic reticulum content. These features can be observed with light microscopy, antibody- or dye-enabled epifluorescent or confocal microscopy, or transmission electron microscopy of PSC-CMs or native cardiomyocytes (as monolayers, clumps, or engineered constructs).

### Transcriptional Characterization

PSC-CMs or native CMs can be interrogated for the expression of transcripts associated with maturity, including cytoskeletal, ion channel, sarcolemmal and sarcoplasmic Ca²⁺ handling, metabolic, and transcriptional regulatory (e.g. transcription factors and miRNA) genes; these transcripts or miRNAs can be assessed via quantitative PCR (qPCR) or RNA sequencing. Sufficient RNAseq benchmarks for immature and “matured” iPSC-CM monolayers or iPSC-CM-containing tissue engineered constructs, as well as fetal, neonatal, and adult tissues can be found in the literature e.g. Van Den Berg et al. (2015). Transcriptome of human foetal heart compared with cardiomyocytes from pluripotent stem cells. Dev. 142, 3231-3238; Churko et al. (2018). Defining human cardiac transcription factor hierarchies using integrated single-cell heterogeneity analysis. Nat. Commun. 9; Feyen et al. (2020). Metabolic Maturation Media Improve Physiological Function of Human iPSC-Derived Cardiomyocytes. Cell Rep. 32; Gentillon et al. (2019). Targeting HIF-1α in Combination with PPARα Activation and Postnatal Factors Promotes the Metabolic Maturation of Human Induced Pluripotent Stem Cell-derived Cardiomyocytes. J. Mol. Cell. Cardiol. 132, 120-135; Giacomelli et al. (2020). Human-iPSC-Derived Cardiac Stromal Cells Enhance Maturation in 3D Cardiac Microtissues and Reveal Non-cardiomyocyte Contributions to Heart Disease. Cell Stem Cell 26, 862-879.e11; Gilsbach et al. (2018). Distinct epigenetic programs regulate cardiac myocyte development and disease in the human heart in vivo. Nat. Commun. 9; Kuppusamy et al. (2015). Let-7 family of microRNA is required for maturation and adult-like metabolism in stem cell-derived cardiomyocytes. Proc. Natl. Acad. Sci. U.S.A 112, E2785-E2794; Mills et al. (2017). Functional screening in human cardiac organoids reveals a metabolic mechanism for cardiomyocyte cell cycle arrest. Proc. Natl. Acad. Sci. U.S.A 114, E8372-E8381; Nakano et al. (2017). Glucose inhibits cardiac muscle maturation through nucleotide biosynthesis. Elife 6, 1-23; Yang et al. (2019). Fatty Acids Enhance the Maturation of Cardiomyocytes Derived from Human Pluripotent Stem Cells. Stem Cell Reports 13, 657-668; and Zhao et al. (2019). A Platform for Generation of Chamber-Specific Cardiac Tissues and Disease Modeling. Cell 176, 913-927.e18; the contents of which are incorporated herein by reference.

### Expressional Characterization

PSC-CMs or adult CMs can be interrogated for protein expression characteristic of previously-benchmarked levels of maturity. Targeted protein detection (corresponding to the genes of interest identified above) can be accomplished by immunoblotting, proteomic profiling, and flow cytometry of either extracellular or intracellular proteins; the former two methods utilize various forms of protein extract, while the latter utilizes one or more collections of live or fixed single cells, labelled with antibodies targeting the proteins of interest. Cai et al. (2019). An Unbiased Proteomics Method to Assess the Maturation of Human Pluripotent Stem Cell-Derived Cardiomyocytes. Circulation Research 125:936-953 provides examples.

Methods of characterizing cardiomyocytes on a functional basis, on a transcriptional basis, or on an expressional basis can be found e.g. in U.S. Pat. No. 10,696,947, the teachings of which are incorporated herein by reference.

### Cell Culture Composition

Significant progress has been achieved towards iPSC-CM maturation in 3D culture models using physical and electrical stimuli and basic co-culture with fibroblasts [Zhao Y, Aschar-Sobbi R, Radisic M, et al (2019) A Platform for Generation of Chamber-Specific Cardiac Tissues and Disease Modeling. Cell 176:913-927.e18; Ronaldson-Bouchard K, Ma S P, Yeager K, et al (2018) Advanced maturation of human cardiac tissue grown from pluripotent stem cells. Nature 556:239-243]. However, without additional optimization of other aspects of culture, there are likely diminishing returns to be found in further developing these factors.

Culture medium optimization has been recently explored as a high-yield avenue for further iPSC-CM maturation. Advances have been made using one- or two-factor experiments. However, there has been little focus on supplementation of cofactors or otherwise functional small molecules whose synthesis would likely be deprioritized in fully mature CMs. Furthermore, pairwise and higher-order interactions have been found to be ubiquitous across biological systems; optimizations of one or two factors at a time fail to model high-level or diverse factor interactions that would occur with the concurrent optimization of higher numbers of factors. At the same time, traditional multifactorial statistical modeling strategies such as design of experiments methodologies that account for interactions cannot practically cover solution spaces comprising many factors.

As detailed further in the Examples, the inventors developed an effective medium for maturing iPSC-CM using a suite of soluble factors comprising metabolic substrates, hormones, cofactors, and other small molecules based on the potential for widescale interactions between factors. Without wishing to be bound by a theory as to the utility of these factors within the claimed cell culture composition, proposed utility of these co-factors is noted below. Cardiomyocytes matured using the developed medium demonstrated improvement in a number of the metrics of maturity (functional, transcriptional and expressional) over commercial gold standards as well as published formulations.

Suitably, the culture medium is a serum-free medium. As is known to persons skill in the art, “serum-free” media is substantially free of serum and lacks whole serum as an ingredient, but may include certain serum-derived products and, in particular, purified forms of albumin.

Suitably, culture medium composition may be prepared using a chemically-defined serum-free medium. Such mediums are known in the art and are commercially available. Mixtures of one or more chemically specified serum-free media can be employed in the culture medium compositions of the present invention. In a preferred embodiment, culture medium compositions according to the present disclosure are suitably prepared using Media 199 (available e.g. from Sigma-Aldrich).

In various embodiments, the culture medium is prepared using basal medium that include one or more of: Medium 199 (M199); Roswell Park Memorial Institute 1640 (RPMI 1640); Dulbecco's Modified Eagle Medium (DMEM); Eagle's Minimum Essential Medium (MEM); Alpha modification MEM (derivative of MEM); Ham's F-12 nutrient mixture; DMEM/F-12; Iscove's Modified Dulbecco's Medium (IMDM); Connaught Medical Research Laboratories (CMRL).

### Putrescine

The composition suitably includes putrescine, a secondary metabolite that has polyamine effects on cell growth, proliferation, and hypertrophy, maintenance of inward rectifying current, protein stabilization and excitation-contraction kinetic modulation. In various embodiments, putrescine is included in a culture medium in an amount of 0.001 mM to 0.03 mM, more preferably 0.01 mM to 0.03 mM, most preferably 0.02 mM to 0.025 mM.

### Metformin

The composition suitably includes metformin, a small molecule that is believed to have utility via increased catabolism through AMPK axis, increased insulin sensitivity, increased lipid catabolism, inhibition of lipotoxicity and Ca2+ regulation. In various embodiments, metformin is included in a culture medium in an amount of 0.001 mM and 0.25 mM, more preferably 0.01 mM and 0.15 mM, most preferably 0.05 mM and 0.10 mM.

### Leukemia Inhibitory Factor (LIF)

The composition suitably includes leukemia inhibitory factor (LIF), which is associated with cardiomyocyte hypertrophy. In various embodiments, LIF is included in a culture medium in an amount of 0.1 ng/mL and 10 ng/mL, more preferably 2 ng/mL to 8 ng/mL, most preferably about 6 ng/mL.

### Carbon Source or Central Carbon Metabolism Inhibitor

The composition suitably includes a high-flux carbon source and/or central carbon metabolism inhibitor.

In one embodiment, the high-flux carbon source is a lipid mixture. The lipid mixture suitably includes cholesterol and one or more lipids selected from: linoleic acid, linolenic acid, myristic acid, oleic acid, palmitic acid, palmitoleic acid and stearic acid. The lipid mixture provides an oxidative carbon source, while cholesterol maintains specialized membrane functionality and is a steroid precursor. Suitably, the lipid mixture may be Chemically Defined Lipid (CDL) Concentrate (available from ThermoFisher Scientific). For example, the CDL concentrate can contain a total of 1 to 3 g/L of lipids. The lipids can be selected from the group consisting of arachidonic acid, cholesterol, DL-alpha-tocopherol acetate, linoleic acid, linolenic acid, myristic acid, oleic acid, palmitic acid, palmitoleic acid, stearic acid and Tween 80™. The concentration of arachidonic acid can be from 1.5 to 2.5 mg/L. The concentration of cholesterol can be from 165 to 275 mg/L. The concentration of DL-alpha-tocopherol acetate can be from 52 to 88 mg/L. The concentrations of linoleic acid, linolenic acid, myristic acid, oleic acid, palmitic acid, palmitoleic acid, and stearic acid can be independently from 7.5 to 12.5 mg/L. The concentration of Tween 80™ can be from 1650 to 2750 mg/L.

In some embodiments, the composition includes lactate, an oxidative carbon source. In various embodiments, lactate is included in a culture medium in an amount of 0.001 mM to 10 mM, more preferably 0.001 mM to 1 mM, most preferably 0.001 mM to 0.10 mM. In some embodiments, the composition does not include lactate.

In some embodiments, the medium contains galactose, a carbon source that inhibits lipotoxicity. In various embodiments, galactose is included in a culture medium in an amount of 0.001 mM to 40 mM, more preferably 10 mM to 30 mM, most preferably 15 mM to 25 mM.

In some embodiments, the medium contains 2-deoxyglucose, which inhibits glycolysis, PPP/nucleotide metabolism, and hexosamine synthesis, and which may exert antiglycolytic synergies with insulin and metformin. In various embodiments, 2-deoxygluclose is included in a culture medium in an amount of 0.001 mM to 20 mM, more preferably 5 mM to 10 mM, most preferably about 6 mM.

Suitably, the composition contains a plurality, and preferably, all of a lipid mixture, lactate and galactose. In some embodiments, the composition further includes 2-deoxyglucose.

In one embodiment, the medium composition contains 2-deoxyglucose, insulin and metformin.

### Metabolites

The culture medium suitably contains one or more further metabolites not responsible for substantial central carbon metabolic flux, which may be selected from creatine, carnitine, taurine, and ethanolamine. In a preferred embodiment, the culture medium contains all of creatine (a local highly-available energy pool, regenerated between contractions), carnitine (transporter of fatty acids within cells), taurine (an antioxidant associated with calcium homeostasis), and ethanolamine (a phospholipid precursor for electroactive membrane structure).

In various embodiments, creatine is included in a culture medium in an amount of 0.001 mM to 6.5 mM, more preferably 0.01 mM to 2 mM, still more preferably about 0.1 mM to 1 mM, and most preferably, about 0.2 mM.

In various embodiments, carnitine is included in a culture medium in an amount of 0.001 mM to 6.5 mM, more preferably 0.01 mM to 2 mM, still more preferably about 0.1 mM to 1 mM, and most preferably, about 0.2 mM.

In various embodiments, taurine is included in a culture medium in an amount of 0.001 mM to 6.5 mM, more preferably 0.01 mM to 2 mM, still more preferably 0.1 mM to 1 mM and, most preferably about 0.5 mM.

In various embodiments, ethanolamine is included in a culture medium in an amount of 0.001 mM to 0.3 mM, more preferably 0.001 mM to 0.1 mM, most preferably 0.001 mM to 0.01 mM.

### Hormones

The culture medium suitable includes at least one and, more preferably a plurality, of the following hormones, (which, again without being bound by a theory of activity, are believed to have the utility noted in brackets): insulin (growth and differentiation signal); insulin-like growth factor (cell proliferation, growth, and survival); triiodothyronine (physiological cardiac hypertrophy and maturation); hydrocortisone (glucocorticoid influence on cardiac maturation); neuregulin (cardiac maturation, structural development and maintenance, Ca²⁺ homeostasis); and human growth hormone (cell hypertrophy, protein synthesis, oxidative metabolism, attenuation of glycolytic pathways).

In various embodiments, insulin is included in a culture medium in an amount of 0.75 to 1.25 g/L, preferably 0.8 to 1.2 g/L, more preferably 0.85 to 1.15 g/L, still more preferably 0.9 to 1.1 g/L and most preferably 1.0 g/L.

In various embodiments, insulin-like growth factor is included in a culture medium in an amount of 0.001 ng/mL to 100 ng/mL, more preferably 1 ng/mL to 50 ng/mL, still more preferably 10 ng/mL to 40 ng/mL and most preferably, about 33 ng/mL.

In various embodiments, triiodothyronine is included in a culture medium in an amount of 0.001 ng/mL to 80 ng/mL, more preferably 0.1 ng/mL to 20 ng/mL, still more preferably about 1 ng/mL to 10 ng/mL and most preferably, about 8 ng/mL.

In various embodiments, hydrocortisone is included in a culture medium in an amount of 0.001 ng/mL to 80 ng/mL, more preferably 0.1 ng/mL to 20 ng/mL, still more preferably about 1 ng/mL to 10 ng/mL and most preferably, about 8 ng/mL.

In various embodiments, neuregulin is included in a culture medium in an amount of 0.001 ng/mL to 5 ng/mL, more preferably 0.01 ng/mL to 3 ng/mL, still more preferably about 0.01 ng/mL to 1 ng/mL and most preferably, about 0.1 ng/mL.

In various embodiments, human growth hormone is included in a culture medium an amount of 0.001 ng/mL to 100 ng/mL, more preferably 0.001 ng/mL to 10 ng/mL, still more preferably about 0.001 ng/mL to 1 ng/mL and still more preferably, <0.1 ng/mL.

### Proteins Involved in Cell Homeostasis

Suitably, the culture medium further comprises one or more proteins involved in cell homeostasis.

Suitably, the culture medium comprises albumin, which is believed to have utility as a hormone carrier and lipid carrier. In various embodiments, albumin is included in a culture medium an amount of 1-3 wt %, more preferably 1-2 wt %, still more preferably 1.25-1.35 wt % and still more preferably, about 1.3 wt %.

Suitably, the culture medium includes transferrin, which is believed to have utility in iron homeostasis. In various embodiments, transferrin is included in a culture medium an amount of 0.4 to 0.7 g/L, preferably 0.45 to 0.65 g/L, more preferably 0.5 to 0.6 g/L and most preferably 0.55 g/L.

### Other Molecules

In some embodiments, the culture medium comprises one or more other small molecules.

In one embodiment, the medium comprises one or more biological antioxidants. In one embodiment the medium contains β-mercaptoethanol. In various embodiments, β-mercaptoethanol is included in a culture medium an amount of 0.001 mM and 0.1 mM, more preferably about 0.01 mM.

### Supplemental Elements

The culture medium may suitably contain one or more supplemental elements. Without limiting the generality of the foregoing, in one embodiment, the culture medium comprises selenium (a cofactor for redox processes), which can suitably be used in the form of a selenite anion. In various embodiments, selenium is included in a culture medium in an amount of 0.4 to 1.0 mg/L, preferably 0.5 to 0.9 mg/L, more preferably 0.55 to 0.8 mg/L, still more preferably 0.6 to 0.75 mg/L and most preferably 0.67 mg/L.

### Other Components

The medium may contain other components. Without limiting the generality of the foregoing, these additional components may be those found in a commercially available chemically defined medium, such as Medium 199.

In one embodiment, the medium further contains one or more antibiotics. Antibiotic mixtures, including combinations, are commercially available. In some embodiments, the medium includes penicillin, streptomycin and/or gentamicin, which may be used at the following concentrations: 50 to 100 I.U./mL penicillin; 50 to 100 μg/mL streptomycin; 25 to 75 μg/mL gentamicin.

### Exemplary Compositions

In some embodiments, the medium composition comprises a plurality, and in one embodiment all, of the factors identified in Table 1. In various embodiments, one or more of the plurality or all of the factors identified are included at the levels identified in Table 1.

| Component | Level | |
|---|---|---|
| 2-deoxyglucose | 0-20 mM or 0-3280 mg/L | |
| Lactate | 0-10 mM or 0-891 mg/L | |
| Lipid mixture | 0-3 | g/L |
| Galactose | 0-25 mM or 0-4504 mg/L | |
| Creatine | 0-6.4 mM or 0-839 mg/L | |
| Carnitine | 0-6.4 mM or 0-1032 mg/L | |
| Taurine | 0-6.4 mM or 0-801 mg/L | |
| Insulin-Transferrin-Selenium (ITS) | 0-3.2 | X |
| Albumin | 1-3 | wt. % |
| β-mercaptoethanol | 0-0.1 | mM |
| Putrescine | 0-0.0226 | mM |
| Ethanolamine | 0-0.0316 | mM |
| Triiodothyronine | 0-80 | ng/mL |
| Leukemia inhibitory factor | 0-6 | ng/mL |
| Insulin-like growth factor | 0-100 | ng/mL |
| Human growth hormone | 0-100 | ng/mL |
| Hydrocortisone | 0-79.1 | ng/mL |
| Neuregulin | 0-3.2 | ng/mL |
| Metformin | 0-0.250 | mM |

In one embodiment, the composition includes some of, and preferably all of, the factors identified in Table 2, in one embodiment, at the level provided in Table 2.

TABLE 2

| Component | Level | |
|---|---|---|
| 2-deoxyglucose | 6.32 ± 25% mM (1074 ± 25% mg/L) | |
| Lipid mixture | 2.2-2.6 | g/L |
| galactose | 25 ± 25% mM (4504 ± 25% mg/L) | |
| Creatine | 0.2 ± 25% mM (26 ± 25% mg/L) | |
| Carnitine | 0.2 ± 25% mM (32 ± 25% mg/L) | |
| Taurine | 0.5 ± 25% mM (63 ± 25% mg/L) | |
| Insulin | 1.0 ± 25% | g/L |
| Transferrin | 0.55 ± 25% | g/L |
| Selenium | 0.67 ± 25% | mg/L |
| Albumin | 1.32 ± 25% | wt. % |
| β-mercaptoethanol | 0.0101 ± 25% | mM |
| putrescine | 0.0226 ± 25% | mM |
| ethanolamine | 0.00157 ± 25% | mM |
| Triiodothyronine | 8 ± 25% | ng/mL |
| Leukemia inhibitory factor | 6 ± 25% | ng/mL |
| Insulin-like growth factor | 32.6 ± 25% | ng/mL |
| Hydrocortisone | 7.91 ± 25% | ng/mL |
| Neuregulin | 0.1 ± 25% | ng/mL |
| Metformin | 0.0791 ± 25% | mM |

The culture medium may further include a physiologically acceptable saline solution. In one embodiment an isotonic saline solution.

Cell culture compositions as provided herein may be in solid or liquid form. In one embodiment, the culture medium is provided in a liquid form.

Culture compositions as provided herein, which may be in solid or liquid form, may be added to commercially available culture mediums or homemade medium formulations to improve the cardiomyocyte maturing properties of the culture medium.

### Matured Cardiomyocytes

Cardiovascular disease is the single most common cause of death in developed countries, and a leading cause for cardiovascular disease is myocardial infarction (MI). In addition to acute mortality, patients surviving MI are at serious risk for complications due to cardiomyocyte (CM) death, including subsequent MI, heart failure, arrhythmia, and mechanical tissue failure modes including wall rupture or cardiac tamponade [Elbadawi A, Elgendy I Y, Mahmoud K, et al (2019) Temporal Trends and Outcomes of Mechanical Complications in Patients With Acute Myocardial Infarction. JACC Cardiovasc Interv 12:1825-1836; Huber C A, Meyer M R, Steffel J, et al (2019) Post-myocardial Infarction (MI) Care: Medication Adherence for Secondary Prevention After MI in a Large Real-world Population. Clin Ther 41:107-117.]

As post-infancy CM proliferation is minimal [see Yester J W, Kuhn B (2017) Mechanisms of Cardiomyocyte Proliferation and Differentiation in Development and Regeneration. Curr Cardiol Rep 19:13; Bergmann O, Bhardwaj R D, Bernard S, et al (2009) Evidence for Cardiomyocyte Renewal in Humans. Science (324) 5923:98-102] and methods to reinitiate the cell cycle in situ [see Mohamed TMA, Ang Y-S, Radzinsky E, et al (2018) Regulation of Cell Cycle to Stimulate Adult Cardiomyocyte Proliferation and Cardiac Regeneration. Cell 173:104-116.e12] are in their infancy intrinsic regeneration is unlikely to reach clinical implementation in coming years.

Exogenous supplementation with CMs via injection [Romagnuolo R, Laflamme M A (2017) Programming cells for cardiac repair. Curr Opin Biotechnol 47:43-50] or the use of engineered patches [Montgomery M, Ahadian S, Davenport Huyer L, et al (2017) Flexible shape-memory scaffold for minimally invasive delivery of functional tissues. Nat Mater 16:1038-1046], both using induced pluripotent stem cell-derived cardiomyocytes (iPSC-CMs) provide alternative myocardial remusculatization approaches. Improving the functionality of the iPSC-CMs prior to transplantation via maturation may reduce the arrhythmia risk of injected iPSC-CM. In this regard, as evidenced in Example 2, culturing cells in a maturation medium as provided herein can reduce spontaneous contractility. In various embodiments, CMs matured according to methods provided herein may be used in therapies for the treatment of cardiovascular diseases or conditions, including therapies involving exogenous supplementation with CMs.

In addition to therapies, iPSC-CMs have utility in cardiac pharmaceutical efficacy screening, both for preclinical screening and personalized efficacy testing; details of such uses can be found e.g. in Matsa E, Burridge P W, Yu K-H, et al (2016) Transcriptome Profiling of Patient-Specific Human iPSC-Cardiomyocytes Predicts Individual Drug Safety and Efficacy Responses In Vitro. Cell Stem Cell 19:311-325; Yang C, Al-Aama J, Stojkovic M, et al (2015) Concise Review: Cardiac Disease Modeling Using Induced Pluripotent Stem Cells. Stem Cells 33:2643-2651; and Knollmann B C (2013) Induced pluripotent stem cell-derived cardiomyocytes: Boutique science or valuable arrhythmia model? Circ Res 112:969-976, the contents of which are incorporated herein by reference.

Cardiotoxicity is a leading reason for clinical trial failure or post-marketing drug recalls. For both efficacy and toxicity profiling, high-fidelity pharmaceutical screening with improved predictive power towards drug efficacy and toxicity than existing animal or culture models is required. Drug responses, including efficacy and toxicity, are emergent effects of interactions across these aspects of physiology, and are therefore subject to the propagation of inaccuracies in a cell model relative to the corresponding tissue in vivo. Current iPSC-CMs are phenotypically immature in all aspects of CM-specific physiology; they do not recapitulate the contractile force, morphology, electrophysiology, calcium handling characteristics, or metabolic profile associated with adult myocardium [GuFo Y, Pu WT (2020) Cardiomyocyte Maturation. Circ Res 126:1086-1106.] For this reason, iPSC-CM drug toxicity models are not predictive of patient responses either individually or in aggregate [Feric N T, Pallotta I, Singh R, et al (2019) Engineered Cardiac Tissues Generated in the Biowire II: A Platform for Human-Based Drug Discovery. Toxicol Sci 172:89-97].

Disease modeling has been successfully accomplished using iPSC-CMs—see e.g. Yang C, Al-Aama J, Stojkovic M, et al (2015) Concise Review: Cardiac Disease Modeling Using Induced Pluripotent Stem Cells. Stem Cells 33:2643-2651; Weber N, Schwanke K, Greten S, et al (2016) Stiff matrix induces switch to pure β-cardiac myosin heavy chain expression in human ESC-derived cardiomyocytes. Basic Res Cardiol 111:68; Wyles S P, Li X, Hrstka S C, et al (2016) Modeling structural and functional deficiencies of RBM20 familial dilated cardiomyopathy using human induced pluripotent stem cells. Hum Mol Genet 25:254-265; Ovchinnikova E, Hoes M, Ustyantsev K, et al (2018) Modeling Human Cardiac Hypertrophy in Stem Cell-Derived Cardiomyocytes. Stem Cell Reports 10:794-807; Wang E Y, Rafatian N, Zhao Y, et al (2019) Biowire Model of Interstitial and Focal Cardiac Fibrosis. ACS Cent Sci 5:1146-1158; and Wang G, McCain M L, Yang L, et al (2014) Modeling the mitochondrial cardiomyopathy of Barth syndrome with induced pluripotent stem cell and heart-on-chip technologies. Nat Med 20:616-23, the disclosures of which are incorporated herein reference. However, its utility in target identification or mechanistic discovery remains limited; due to their immature glycolysis-dominant metabolic profile that renders iPSC-CMs resistant to ischaemic damage, MIs are particularly difficult to model [Hidalgo A, Glass N, Ovchinnikov D, et al (2018) Modelling ischemia-reperfusion injury (IRI) in vitro using metabolically matured induced pluripotent stem cell-derived cardiomyocytes. APL Bioeng 2:026102.; Sebastião M J, Serra M, Pereira R, et al (2019) Human cardiac progenitor cell activation and regeneration mechanisms: Exploring a novel myocardial ischemia/reperfusion in vitro model. Stem Cell Res Ther 10:1-16.]. Thus, in some embodiments, CMs matured according to methods provided herein may be used in pharmaceutical screening, including efficacy and toxicity screens, or disease modeling, including, but not limited to modeling of MI.

### Heading, Incorporation by Reference and Modifications

The section headings used herein are for organizational purposes only and are not to be construed as limiting the subject matter described.

All documents referenced herein are incorporated by reference, however, it should be appreciated that any patent, publication, or other disclosure material, in whole or in part, that is incorporated by reference herein is incorporated only to the extent that the incorporated material does not conflict with definitions, statements, or other disclosure material set forth in this disclosure. As such, and to the extent necessary, the disclosure as explicitly set forth herein supersedes any conflicting material incorporated herein by reference.

It will be understood that numerous modifications thereto will appear to those skilled in the art. Accordingly, the above description, accompanying drawings and examples that follow should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

The embodiments of the invention described above are intended to be exemplary only. The scope of the invention is therefore intended to be limited solely by the scope of the appended claims.

### EXAMPLES

### Example 1. Formulation of Maturation Medium and Characterization of Cells Cultured Using Maturation Medium

The inventors sought to effectively mature iPSC-CM using a suite of soluble factors comprising metabolic substrates, hormones, cofactors, and other small molecules based on the potential for widescale interactions between factors. The general strategy in choosing factors was to mirror the exogenous availability of soluble signals supplied to the myocardium underlying late cardiac development, specifically the perinatal window and early childhood, which co-occurs with a switch to a predominantly oxidative metabolism [Kannan S, Kwon C (2020) Regulation of cardiomyocyte maturation during critical perinatal window. J Physiol 598:2941-2956.; de Carvalho A E T S, Bassaneze V, Forni M F, et al (2017) Early Postnatal Cardiomyocyte Proliferation Requires High Oxidative Energy Metabolism. Sci Rep 7:15434.] This transition to a highly-efficient energetic phenotype may drive maturation in other metrics by increasing the accessible short-term energetic capacity within the cell [Piquereau J, Ventura-Clapier R (2018) Maturation of Cardiac Energy Metabolism During Perinatal Development. Front Physiol 9:1-10.]

To maximize factor synergies and high-order interactions, a newly-developed high dimensional, directed evolution (HD-DE) algorithm that provides an economical means to search over a large factor space [Kim M M, Audet J (2019) On-demand serum-free media formulations for human hematopoietic cell expansion using a high dimensional search algorithm. Commun Biol 2:1-11] was applied; by combining a set of 17 independent soluble factors at 5 levels each of dosage, a traditional full factorial screen would require c.a. 763 billion runs to cover the solution space. By use of the HD-DE methodology, a high-performing custom formulation was iteratively developed after 4 iterative generations comprising 169 unique formulations. These formulations were then tested for a variety of maturation metrics (morphology, cytoskeletal and sarcoplasmic structure, contractile force, Ca²⁺ handling and drug responses, and metabolic, transcriptomic and proteomic profiling) against existing gold-standard maturation medium formulations.

A medium formulation was identified that best enhances PSC-CM maturity to maximize multiple functional performance metrics against existing formulations. This formulation demonstrated unmatched advancement of a variety of morphological and functional metrics of maturity including the detection of Ca²⁺ transient modulators, contractility, metabolism across both 2D and 3D culture platforms.

### Methods

No statistical methods were used to predetermine sample size. Experimental wells were randomized, but blinding of conditions could not be used during experiments or outcome assessment due to physical differences (e.g., medium colour) between the treatments used and the morphological differences in the resulting cells.

### DESIGN of an Iterative Algorithmic Search for Optimized Maturation Medium

The Custom maturation medium was non-prescriptively optimized in a large solution space (˜763 billion discrete formulations for an analogous 5¹⁷ factorial experimental design), using the uncoupled:control ratio (UCR; the ratio of FCCP-treated to baseline oxygen consumption rate) as a novel objective metric. This design philosophy allowed for agnostic testing and the evolution of emergent interactions between multiple defined soluble factors within a formulation; several of these factors had not been surveyed previously for efficacy toward PSC-CM maturation. Additionally, M199-based formulations were used exclusively for two reasons: firstly, the ionic profile of M199 is closer to that of human plasma than DMEM- or RPMI 1640-based media, especially in [Ca²⁺], for which homeostasis is vital to developing CMs; and secondly, that M199 contains a much more diverse source of cofactors and vitamins than either basal DMEM or RPMI 1640 media. For the latter consideration, the use of a defined formulation negates the possibility of serum providing these essential components, especially in a highly-specialized cell such as a maturing CM, which may outsource many non-core biosynthetic and secondary metabolic pathways to other tissues of the body.

The formulation search was performed using an existing iterative high-dimensional, directed evolution (HD-DE) decision tree algorithm programmed in MATLAB [Kim M M, Audet J (2019) On-demand serum-free media formulations for human hematopoietic cell expansion using a high dimensional search algorithm. Commun Biol 2:1-11]; the script was modified for R2018a version compatibility, for the use of 17 variable factors, and for duplicate runs. Briefly, the algorithm generates formulation candidate vectors within a defined number of runs, factors, and levels of said factors. Maximal coverage during the initial generation is generated using a Sobol quasi-random distribution (e.g., minimizing discrepancy). After a generation is produced, objective metric scores are reported, theoretically forming a rough Pareto distribution. Formulations within 10% of the top scorer are chosen to continue in the next generation, while poor-scoring formulations are either replaced with randomly-generated vectors, or with offspring vectors from the intergenerational memory of top scorers. The process is formally terminated when the median candidate of the Pareto-ranked generation does not improve by at least 10% after 3 consecutive generations.

### Materials

For iterative formulation candidate screening and characterization of the Custom formulation, soluble components used were 2-deoxyglucose (DXG498; BioShop), sodium L-lactate (L7022; Sigma), Chemically Defined Lipid (11905031; Gibco), D-galactose (GAL500; BioShop), creatine monohydrate (CREE200; BioShop), L-carnitine hydrochloride (C0283; Sigma), taurine (TAU303; BioShop), insulin-transferrin-selenium (41400045; Gibco), bovine serum albumin fraction V (10735078001; Roche), β-mercaptoethanol (M3148; Sigma), putrescine dihydrochloride (PUT001; BioShop), ethanolamine hydrochloride (E6133; Sigma), triiodothyronine (T6397; Sigma), recombinant insulin-like growth factor 1 (PHG0071; Gibco), recombinant leukemia inhibitory factor (SRP3316; Sigma), growth hormone (869008; Millipore Sigma), hydrocortisone (H0888, Sigma), and recombinant neuregulin 1β2 (ab73753; Abcam), metformin hydrochloride (ICN15169101; MP Biomedicals), in a base of Medium 199 (M4350; Sigma).

Gold-standard controls used were iCell cardiomyocytes maintenance medium (CMM-100-120-001; Cellular Dynamics), cardiomyocyte maintenance medium (05020; STEMCELL Technologies), and RPMI 1640 (R8758; Sigma) with 1×B27 supplement (17504044; Gibco).

For immunofluorescent staining, anti-α-actinin (ab9465; Abcam) and anti-connexin 43 (ab217676; Abcam) were used, as well as phalloidin-tetramethylrhodamine B isothiocyanate, (P1951; Sigma), and DAPI (62248; Thermo Scientific). For respirometry, a DMEM-based XF medium (103334; Agilent) was used, with oligomycin (04876; Sigma), FCCP (15218; Cayman Chemical), antimycin A (A8674; Sigma), and rotenone (NC0779735; Cayman Chemical) used as inhibitors. Unless otherwise noted, all other materials were obtained from Sigma.

### Cells

An iterative formulation search, and subsequent characterization of iPSC-CM cultured in the chosen final maturation medium candidate (“Custom”) was performed using the Personal Genome Project of Canada-17 iPSC line (PGPC17), which have been extensively characterized across a wide range of differentiation pathways [Hildebrandt M R, Reuter M S, Wei W, et al (2019) Precision Health Resource of Control iPSC Lines for Versatile Multilineage Differentiation. Stem Cell Reports 13:1126-1141.]

Cells were frozen in clumps in mTeSR (STEMCELL Technologies) containing 10% DMSO, and thawed and cultured when ready for differentiation. Cells were cultured in mTeSR on Geltrex (Thermo-Fisher; LDEV-free, hESC-qualified; incubated at 1:100 in DMEM for 1 h on TCPS plates) and passaged in clumps using ReLeSR (STEMCELL Technologies), all according to manufacturer guidelines. Cells were passaged at least two times after thawing before being expanded for differentiation. PSC-CM differentiation was performed using the Cardiomyocyte Differentiation Kit (STEMCELL Technologies) according to manufacturer directions, with cell seeding densities optimized for each cell line (between 4-8×10⁵ cells well⁻¹ in a 12-well dish, previously coated with 1:100 Matrigel (Corning) in DMEM). Cells were cultured post-differentiation for 2-4 days before reseeding for maturation experiments. Obtained monolayers in 12-well format were dissociated within 1 week of differentiation by incubation for 1 h at 37° C. in 1 mL Hanks buffer, containing 200 U mL⁻¹ collagenase Type II (Worthington), with 0.5 mL TryPLE Select (Gibco) added for 15 additional minutes. Cells were centrifuged 5 min at 300×g and resuspended in RPMI+B27 for 1 d culture before applying treatments.

### Formulation Screening

Soluble factors used in the iterative screening and final Custom formulation (Table 3) were selected with the goal of providing a range of substrates, cofactors, and hormones that would not necessarily be created by a maturing or mature CM but which would be conducive to increased biosynthesis and hypertrophy, oxidative metabolism to fuel highly-energetically demanding physiological processes, or activation of pathways implicated in maturation of myocardium or other functional tissues in vivo. In all, 169 unique formulations were queried over 4 generations of varying size, using the Seahorse XFe96 mitochondrial stress test to interrogate metabolic function.

Cells were dissociated from differentiation wells and seeded at 8×10⁴ cells well⁻¹ in CM support medium on Matrigel-coated XFe96 monolayer plates (Agilent Technologies). Culture medium was changed the next day to the well's respective treatment. Cells were then cultured 21 days in 60 μL medium well⁻¹, with full medium changes every second day for 3 weeks in either duplicates of a formulation candidate, or one of four wells of gold-standard STEMCELL Technologies Cardiomyocyte Maintenance Medium. The four corner wells of the plate were additionally left unseeded as internal measurement controls according to standard XFe96 manufacturer guidelines.

### Respirometry

Cells were equilibrated 45 min before metabolic characterization in initial volume 150 μL Seahorse XF base medium (103334-100, Agilent Technologies) containing additional (in mmol L⁻¹) glucose (5), pyruvate (1), glutamine (1), sodium lactate (5), and 1× Chemically Defined Lipid, to best supply the oxidative substrate flexibility of mature CMs [Pascual F, Coleman RA (2016) Fuel availability and fate in cardiac metabolism: A tale of two substrates. Biochim Biophys Acta-Mol Cell Biol Lipids 1861:1425-1433.] A mitochondrial stress test was performed with sequential injections of 25 μL each (final concentrations): oligomycin (2.5 μmol L⁻¹), carbonyl cyanide-4-phenylhydrazone (FCCP; 1 μmol L⁻¹ for iterative testing, or either 0.20, 0.40, 0.50, 0.65, 0.80, or 1.00 μmol L⁻¹ for characterization of iPSC-CMs cultured in Custom medium), antimycin A and rotenone (2.5 μmol L⁻¹ each). Due to the high specific oxidative flux of CMs, a modified measurement protocol to avoid hypoxia was employed [Readnower R D, Brainard R E, Hill B G, Jones SP (2012) Standardized bioenergetic profiling of adult mouse cardiomyocytes. Physiol Genomics 44:1208-1213]; cells were measured for 2 rather than 3 cycles at each step, with the minimum measurement time of 2 min.

Respirometric measurements were normalized to cell numbers per well. Each well was fixed in 2% formalin for 5 min, washed 3 times with Ca²⁺ and Mg²⁺-free PBS, stained with Hoechst (1 μg mL⁻¹) in PBS without for 5 min, washed 3 additional times, and imaged using an IX71 inverted widefield fluorescent microscope (Olympus Corporation) with a FITC filter. Cell quantifications were performed by performing an automated count of nuclei using ImageJ 1.52p (NIH), by sequential use of the standard Otsu threshold, watershed segmentation, and particle count functions. Measurements were compared between treatments using a 1-way ANOVA followed by Tukey's HSD where indicated.

### Ca²⁺ Imaging

Cells cultured in 12-well plate format were dissociated as described above and replated in single-cell format on 96-well plates with a #1.5 coverslip bottom treated with 1:100 Matrigel, and cultured in their treatment medium to recover for 2 days before imaging. Frozen dry vials of 50 μg Fluo-4 AM (Thermo-Fisher) were reconstituted with 50 μL DMSO containing 20% (w/v) Pluronic F-127. Cells were treated with 50 μL well⁻¹ Fluo-4 AM solution, diluted 1:100 in Tyrode's solution, for 30 min at RT. Cells were rinsed twice with Tyrode's solution and maintained up to 2 h thereafter in 100 μL Tyrode's solution, containing 5 μmol L⁻¹ S-blebbistatin (Toronto Research Chemicals) to prevent movement artifact.

Ca²⁺ imaging was performed on a Fluoview FV3000 (Olympus Corporation) confocal microscope heated to 37° C. Linescan measurements with 488 nm excitation and collection from 505-550 nm at 1% laser power and c.a. 500 V at the HSD were taken at the 20× objective and 2× zoom with spatial and temporal resolutions of 0.62 μm and c.a. 1.2-1.8 ms respectively. Cells were subjected to steady-state stimulation at 1 Hz monophasic square-wave impulses of 2 ms and 40 V at approximately 0.6 cm using custom-made graphite electrodes using an S48 physiological stimulator (Grass Technologies, Warwick, RI). For monolayer sarcoplasmic reticulum (SR) capacity experiments, cells were paced at 1 Hz at steady-state before consecutive additions of verapamil (20 μmol L⁻¹) and caffeine (10 mmol L⁻¹). Fractional SR load upon addition of caffeine was normalized to steady-state transient amplitudes for each cell; comparisons did not assume equal variance and were analyzed by Welch's t-test. Fluo-4 fluorescence measurements were taken using a CCD camera at c.a. 30 Hz framerate on an IX71 inverted epifluorescence microscope (Olympus Corporation, Tokyo, Japan) equipped with a FITC filter cube. Transients were manually extracted using ImageJ and analyzed for kinetics and amplitudes using a custom MATLAB script. Kinetic metrics were compared between treatments using Welch's test and Dunnett's post-hoc test where indicated.

### Immunofluorescence and Confocal Microscopy

Cultured cells were fixed with 2% paraformaldehyde for 10 min at room temperature, followed by 90% ice-cold methanol for 10 min, followed by permeabilization buffer (0.5% Triton X-100, 0.2% Tween-20 in PBS) for 30 min at 4° C. Blocking buffer (5% FBS in permeabilization buffer) was then added and incubated for 1 h at room temperature. Cells were incubated with primary antibodies (α-actinin 1:500, Cx43 1:500) in blocking buffer overnight at 4° C.; incubation in Rhodamine-red-conjugated phalloidin (Sigma) was 1 h at RT. Fluorophore-conjugated secondary antibody staining (anti-rabbit Alexa 488 or anti-mouse Alexa 594; Molecular Probes; 1:800) was performed at room temperature for 1 h in the dark. Nuclear counterstaining was performed using 1 μg ml⁻¹ DAPI at room temperature for 15 min in the dark. Images were acquired using an FV3000 confocal microscope with 405, 488, and 561 nm lasers.

### Contractility Measurement

Contractile kinetics were assessed from phase-contrast videos recorded at ˜20 fps with a 40× objective, using a particle-image velocimetry package for ImageJ [see Tseng, Q. et al. Spatial organization of the extracellular matrix regulates cell-cell junction positioning. Proc. Natl. Acad. Sci. 109, 1506-1511 (2012).]] to quantify relative displacement from the relaxed state between contractions. Individual traces from biological replicates were fitted with a quartic regression using Prism 9 (GraphPad Software Inc., San Diego, USA).

### RNA Sequencing

### Sample Preparation

Total RNA was extracted from iPSC-CMs using the Quiagen RNeasy Micro kit according to manufacturer directions. Bulk RNA was bulk-sequenced at the Princess Margaret Genomics Center (Toronto, Ontario). Reads were aligned to the human reference transcriptome assembly (GR.Ch38) using Salmon [Patro et al. (2017) Salmon provides fast and bias-aware quantification of transcript expression. Nat Methods 14, 417-419.]

### Data Analysis

All data analyses were performed in R. Gene-level read counts were obtained using the Tximeta package [Love et al. (2020) Tximeta: Reference sequence checksums for provenance identification in RNA-seq. PLOS Computational Biology 16(2).]. For the meta-analysis, ComBat-seq [Zhang et al. (2020) ComBat-seq: batch effect adjustment for RNA-seq count data. NAR Genomics and Bioinformatics 2:3] was used to correct for batch effects introduced by the inclusion of samples from different publications. For principal component and t-distributed stochastic neighbor embedding (tSNE) analyses, the raw count matrix was adjusted using the variance stabilizing transformation implemented in the DESeq2 package. PCA and PC-associated GO term enrichment was performed using the pcaExplorer [Marini and Binder (2019) pcaExplorer: an R/Bioconductor package for interacting with RNA-seq principal components. BMC Bioinformatics 20:331] package. tSNE analysis was performed using the Rtsne package, and clusters were determined from tSNE analysis using the Louvain method [Feng et al. (2020) Dimension Reduction and Clustering Models for Single-Cell RNA Sequencing Data: A Comparative Study. Int. J. Mol. Sci. 21(6), 2181], implemented in the bluster package. Differential expression was determined using the DESea2 [Love et al. (2014) Moderated estimation of fold change and dispersion for RNA-seq data with DESeq2. Genome Biology 15:550] package, and Benjamin-Hochberg adjusted p-values <0.05 were considered significant. Gene set enrichment analysis (GSEA) was performed using the GAGE package [Luo et al. (2009) GAGE: generally applicable gene set enrichment for pathway analysis. BMC Bioinformatics 10:161], and terms with Benjamini-Hochberg adjusted p-values <0.05 were considered significant. GO term similarity was determined using the kappa similarity index, and clusters were identified by hierarchical clustering. Pathway enrichment analysis was performed using the GAGE (Luo et al., 2009) package, and pathway diagrams were generated using Pathview [Luo and Brouwer (2013) Pathview: an R/Bioconductor package for pathway-based data integration and visualization. Bioinformatics 29(14):1830-1]. All plots were produced using the ggplot2 and ComplexHeatmap packages.

### Results

### Directed Evolution and Selection of a PSC-CM Maturation Medium

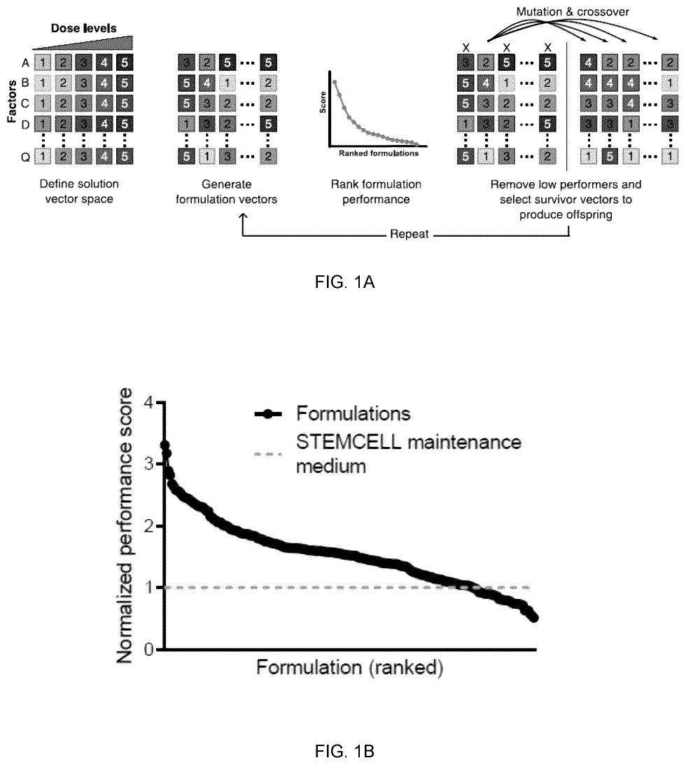

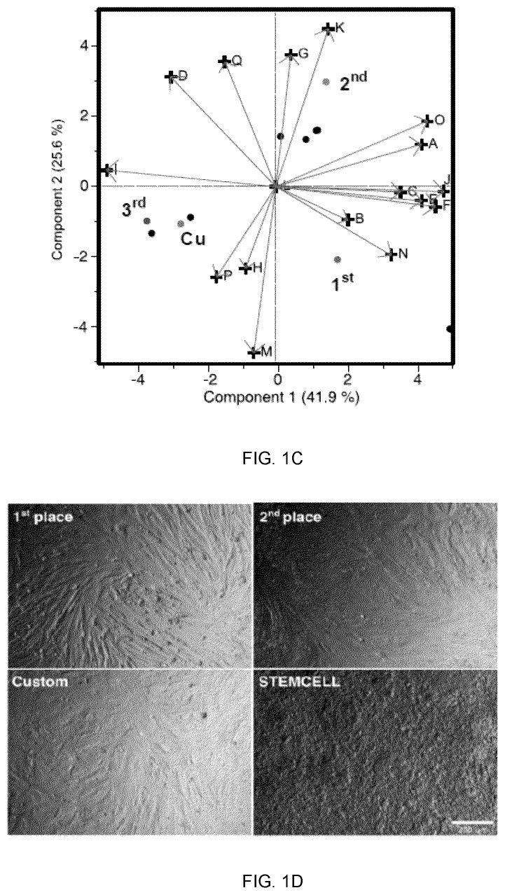

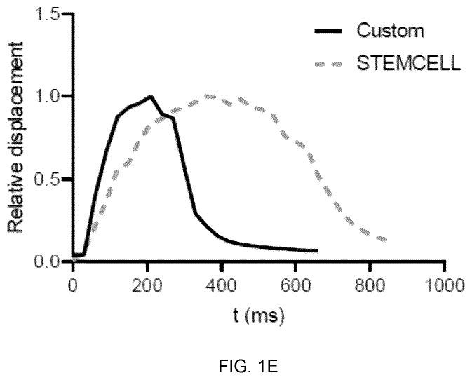

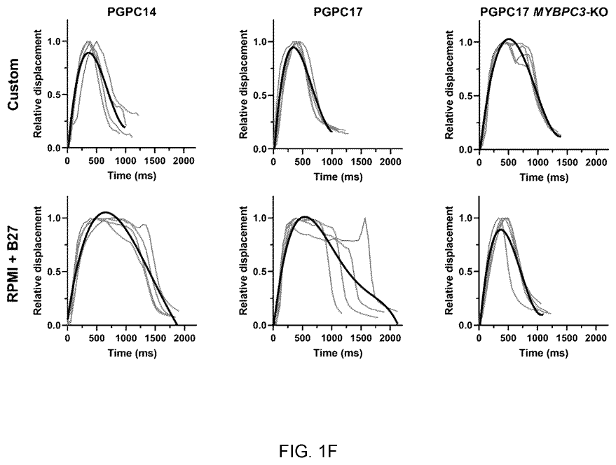

To most efficiently optimize a novel PSC-CM maturation medium in a large solution space, an HD-DE algorithm-driven process was implemented (FIG. 1a), using oxidative uncoupled:control ratio (UCR; or the ratio of pharmaceutically-uncoupled to steady-state oxygen consumption rates) as an objective metric by which each successive generation is scored and compared to a gold-standard control (STEMCELL Cardiomyocyte Maintenance Medium). A total of 17 soluble factors (Table 3) were surveyed at any of 5 doses in a formulation candidate, the lowest of which was a null dose. In all, 169 unique formulations were tested 204 times (FIG. 1b), with each formulation being used for 21 d of culture.

The top 10 performing formulations formed 2 main clusters of composition with 2 outliers (FIG. 1c); substrates and cofactors tended to be loaded into PC1 while signaling-active molecules tended to be loaded mostly into PC2. Most-high performing media demonstrated spontaneous hypertrophy, elongation, and alignment (FIG. 1d). A final formulation for further characterization (“Custom”) (Table 4) was manually defined by including specific dose-factor combinations present in cluster of top-right quadrant of PCA that were absent in the bottom-left quadrant and resulting in a composite of high-performing candidates. Optical contractile traces were obtained using particle image velocimetry; full unpaced contractile cycles for iPSC-CMs (from PGPC17 iPSC line) cultured in Custom medium were completed in approximately half the time of those cultured in STEMCELL medium (FIG. 1e). Similarly, contractile cycles in Custom-cultured monolayers derived from PGPC17 and a health female iPSC-CM line (PGPC14) were accelerated compared to those in cells cultured in RPMI+B27 medium, but slowed with biphasic contraction in a MYBPC3-KO CRISPR-generated PGPC17 mutant iPS line (FIG. 1f).

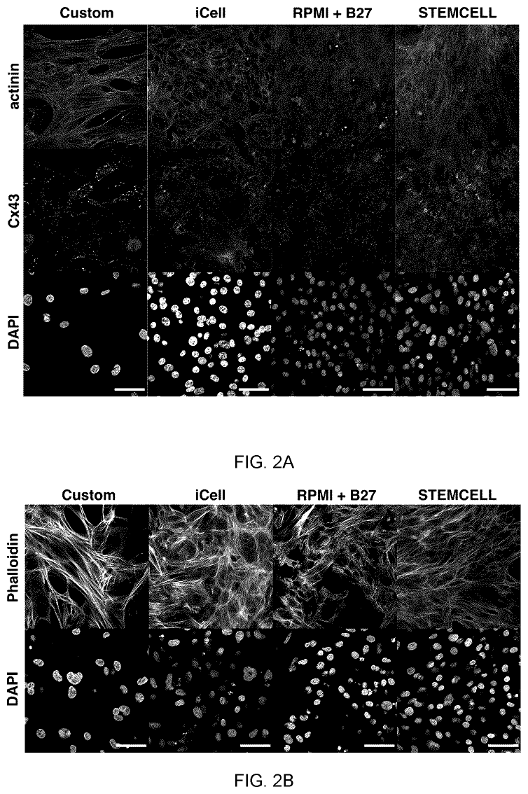

This Custom formulation was confirmed to be a viable and high-performing candidate against gold-standard formulations from STEMCELL, iCell, and the homemade RPMI+B27 cocktail by morphological comparison (FIG. 2) after 6 weeks of culture. Confocal microscopy demonstrated that Custom medium enhanced myofibrillar density, α-actinin striation, and F-actin binding inside cells, and relegation of Cx43 density to cell-cell junctions. (FIG. 2a). the aforementioned contractile performance was supported by the observation of clear sarcomeric striations under phase-contrast in Custom-cultured cells (FIG. 2b).

TABLE 3

| Factor | Levels | |
|---|---|---|
| 2-deoxyglucose | 0-20 | mmol L⁻¹ |
| Lactate | 0-10 | mmol L⁻¹ |
| Chemically-defined lipid | 0-3 | g L⁻¹ |
| Galactose | 0-25 | mmol L⁻¹ |
| Creatine | 0-6.4 | mmol L⁻¹ |
| Carnitine | 0-6.4 | mmol L⁻¹ |
| Taurine | 0-16 | mmol L⁻¹ |
| Insulin | 0-1.25 | g L⁻¹ |
| Transferrin | 0-0.7 | g L⁻¹ |
| Selenium | 0-1 | mg L⁻¹ |
| Albumin | 1-3 | mg mL⁻¹ |
| β-mercaptoethanol | 0-100 | μmol L⁻¹ |
| Putrescine | 0-22.6 | μmol L⁻¹ |
| Ethanolamine | 0-31.6 | μmol L⁻¹ |
| Triiodothyronine | 0-80 | ng mL⁻¹ |
| Leukemia inhibitory factor | 0-6 | ng mL⁻¹ |
| Insulin-like growth factor | 0-100 | ng mL⁻¹ |
| Human growth hormone | 0-100 | ng mL⁻¹ |
| Hydrocortisone† | 0-79.1 | ng mL⁻¹ |
| Neuregulin | 0-3.2 | ng mL⁻¹ |
| Metformin | 0-250 | μmol L⁻¹ |

TABLE 4

| Factor | Level | |
|---|---|---|
| 2-deoxyglucose | 6.32 | mmol L⁻¹ |
| Chemically-defined lipid | 0.33 | X |
| Galactose | 25 | mmol L⁻¹ |
| Creatine | 0.2 | mmol L⁻¹ |
| Carnitine | 0.2 | mmol L⁻¹ |
| Taurine | 0.5 | mmol L⁻¹ |
| Insulin | 1.0 | g L⁻¹ |
| Transferrin | 0.55 | g L⁻¹ |
| Selenium | 0.67 | mg L⁻¹ |
| Albumin | 1.32 | mg mL⁻¹ |
| β-mercaptoethanol | 10.1 | μmol L⁻¹ |
| Putrescine | 22.6 | mg L⁻¹ |
| Ethanolamine | 1.57 | μmol L⁻¹ |
| Triiodothyronine | 8 | ng mL⁻¹ |
| Leukemia inhibitory factor | 6 | ng mL⁻¹ |
| Insulin-like growth factor | 32.6 | ng mL⁻¹ |
| Hydrocortisone | 7.91 | ng mL⁻¹ |
| Neuregulin | 0.1 | ng mL⁻¹ |
| Metformin | 79.1 | μmol L⁻¹ |

Physiological Characterization of Matured iPSC-CMs

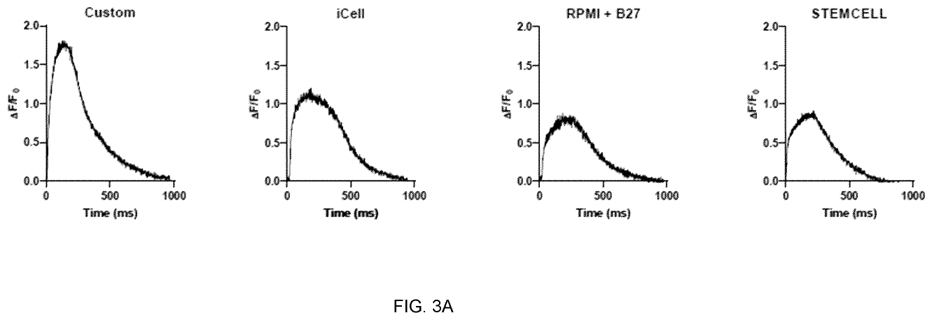

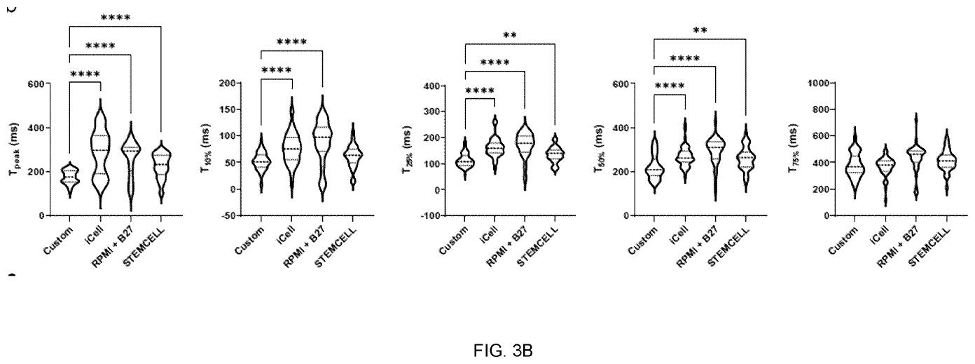

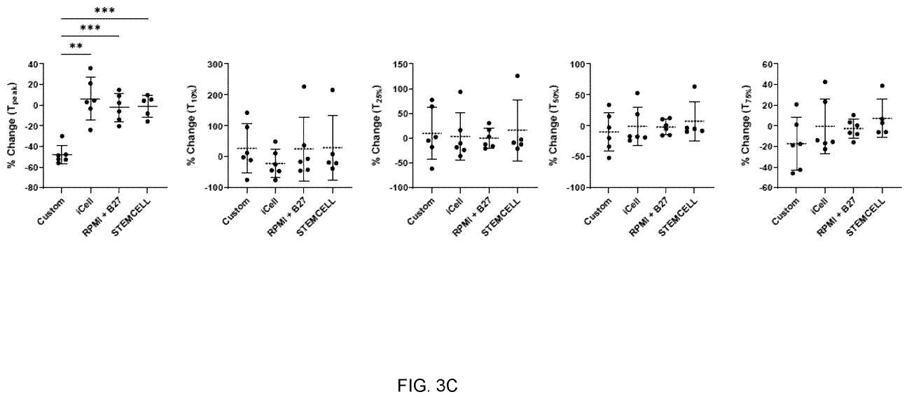

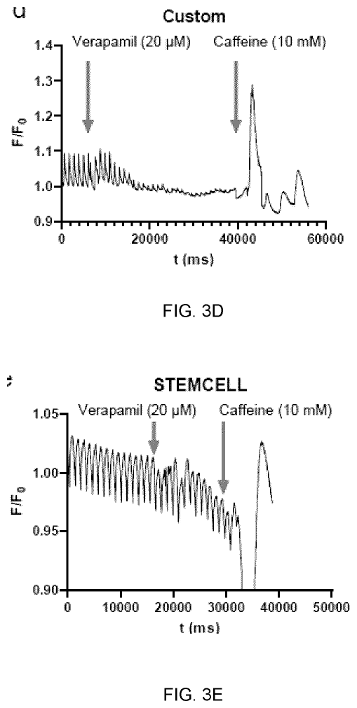

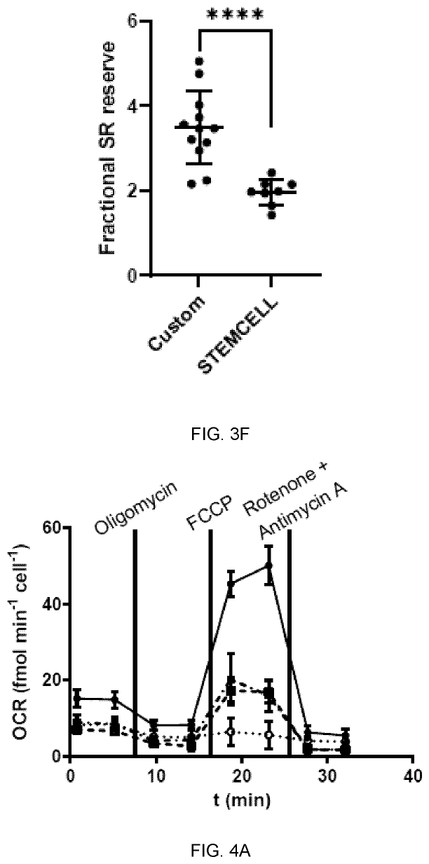

Custom medium-treated iPSC-CMs demonstrated differential Ca²⁺ handling (FIG. 3) compared to gold-standard formulations at steady-state (FIG. 3a) after 6 weeks of culture as assessed by Fluo-4 Ca²⁺ tracking, including both onset and decay kinetic metrics (FIG. 3b); time to peak amplitude was significantly lower than control conditions, as were decay constants of 25% and 50%, while Custom-treated cells also showed significantly shorter 10% decay constant than iCell- and RPMI+B27-treated cells. Under pharmacological manipulation (i.e., stimulation with isoproterenol), Custom-treated iPSC-CMs demonstrated a significantly-accelerated upstroke time (FIG. 3c) compared to all gold-standard conditions. During a physiological SR challenge, where cells paced at 1 Hz were treated with verapamil to inhibit the L-type current before a bolus of caffeine was administered (FIG. 3d-e), Custom-treated cells displayed nearly 2-fold higher peak caffeine-induced transient amplitude normalized to steady-state amplitude than cells cultured in STEMCELL medium (FIG. 3f).

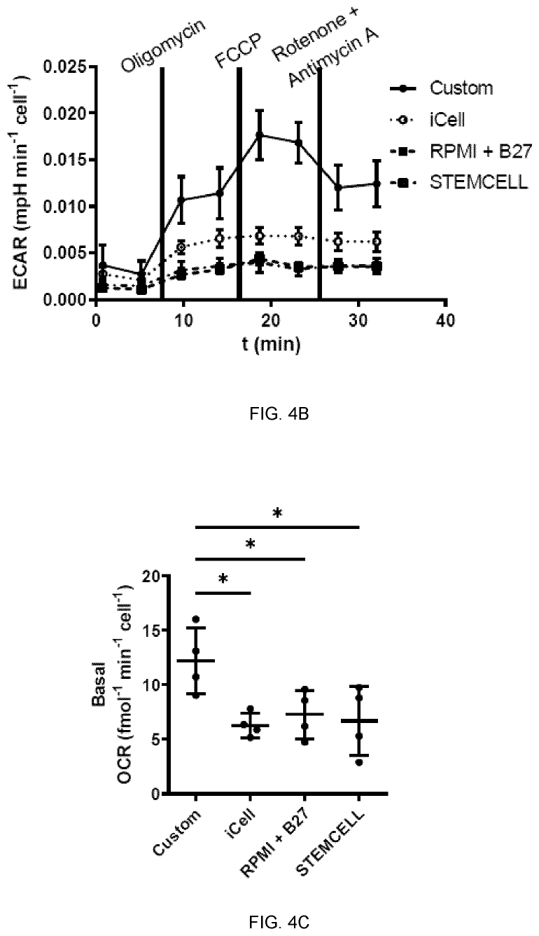

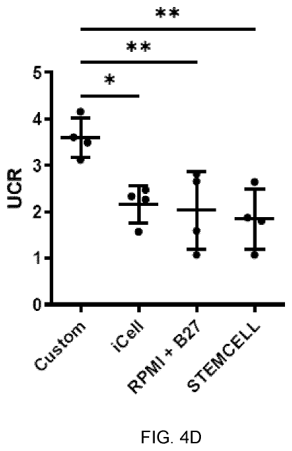

Finally, Custom-treated iPSC-CMs at 6 weeks demonstrated stratification from gold-standard formulations in metabolic profiling using Seahorse XFe96 respirometry (FIG. 4a-b). Specifically, the basal oxygen consumption rate outpaced those of the gold-standard control treatments when normalized to a per-cell basis (FIG. 4c), while the FCCP-enabled uncoupled/control ratio of the Custom treatment was significantly higher than those of the iCell and STEMCELL control treatments (FIG. 4d).

Omics Profiling of Matured iPSC-CMs

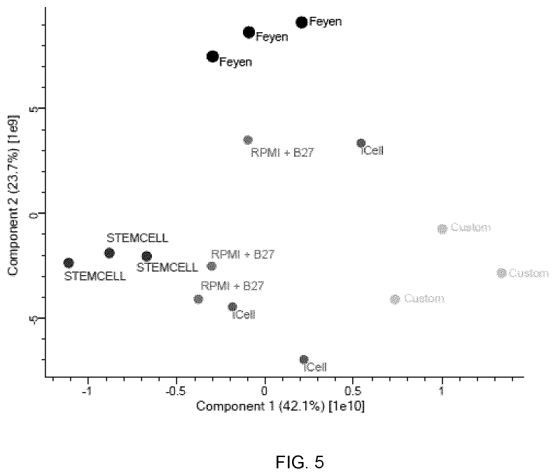

Proteomic analysis was conducted on iPSC-CMs after 6 weeks treatment in Custom, iCell, RPMI+B27, and STEMCELL media, as well as maturation media designed by Feyen et al. [Feyen D A M, McKeithan W L, Bruyneel A A N, et al (2020) Metabolic Maturation Media Improve Physiological Function of Human iPSC-Derived Cardiomyocytes. Cell Rep 32:107925.] (FIG. 5). Principal component analysis (PCA) demonstrated the greatest degree of stratification (i.e., the first PC) on an axis from the STEMCELL to the Custom condition, while PC2 roughly separated the Feyen medium from the other four conditions. Both PC loadings included metabolic and mitochondrial-specific proteins; the positive direction of PC1 also heavily featured sarcoplasmic synthesis and maintenance ontologies, ionoregulatory proteins, and membrane trafficking components, while the positive direction of PC2 heavily featured cytoskeletal proteins associated with contractility. As suggested by the PCA, head to head comparisons revealed higher levels of similarity between Custom, iCell, and Feyen medium conditions than to RPMI+B27 or STEMCELL conditions. Both Custom and Feyen media shared higher expression, relative to the other media, of tricarboxylic acid cycle- and electron-transport chain-related proteins, as well as various proteins involved in the antioxidant response. Custom medium exhibited downregulation from the Feyen medium of several cardiac troponins, cardiac actin, and certain electron transport chain proteins, however also showed significant upregulation of many Ca²⁺-related genes (e.g. SERCA2a and phospholamban), the intercalated disc proteins desmin and plakin, various ER/SR and Golgi-related maintenance and trafficking proteins, and cardiac α-actinin. Both Custom and Feyen media showed down-regulated proteins associated with proliferation, glycolysis, migration, and non-cardiac-specific cytoskeleton.

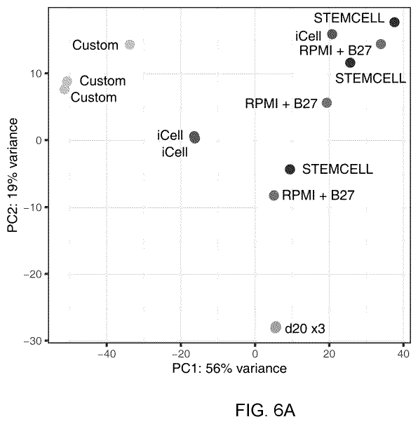

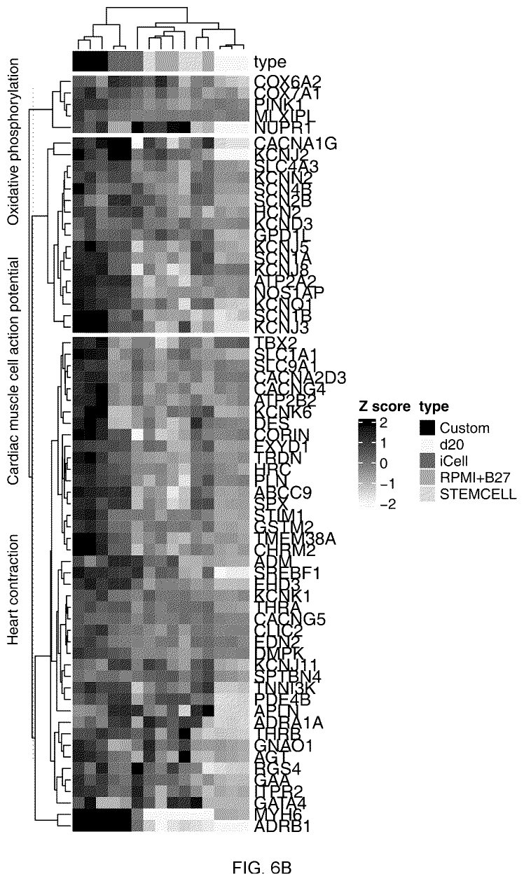

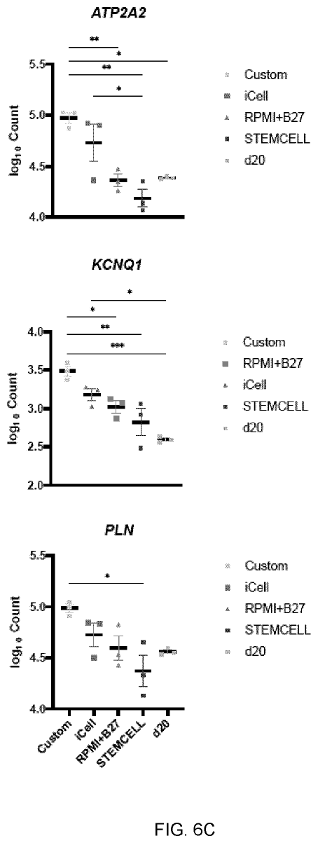

RNA sequencing was used to evaluate the transcriptomic profiles of iPSC-CMs treated with Custom or gold standard maintenance media to help identify targets for further physiological analysis. PCA was used to compare the overall transcriptional profiles of each medium relative to day 20 (d20) controls, demonstrating that Custom-treated cells were highly divergent from both controls and other media (FIG. 6a). To further examine general transcriptomic effects, the number of genes displaying significant differential expression relative to control was quantified for each medium. In agreement with PCA, Custom medium induced differential expression of the largest number of genes (FIG. 6b). Many of the genes uniquely upregulated in Custom-treated cells were associated with general cardiomyocyte functions, including the cardiac action potential, heart contraction and oxidative phosphorylation (FIG. 6c). Importantly, a number of cardiac maturity-specific genes with documented low expression in iPSC-CMs were significantly upregulated in Custom-treated cells (FIG. 6d), including cytoskeletal genes such as MYH7 and ML2, various electrophysiological genes including cardiac sodium and potassium channels, DHPR calcium channels, sarcoplasmic reticulum genes encoding SERCA2a and phospholamban, β-adrenergic response-associated genes, intercalated disc and sarcolemmal organization genes such as caveolins, TMEMs, and desmin, and genes associated with oxidative metabolism, fatty acid transport, and the antioxidant response.

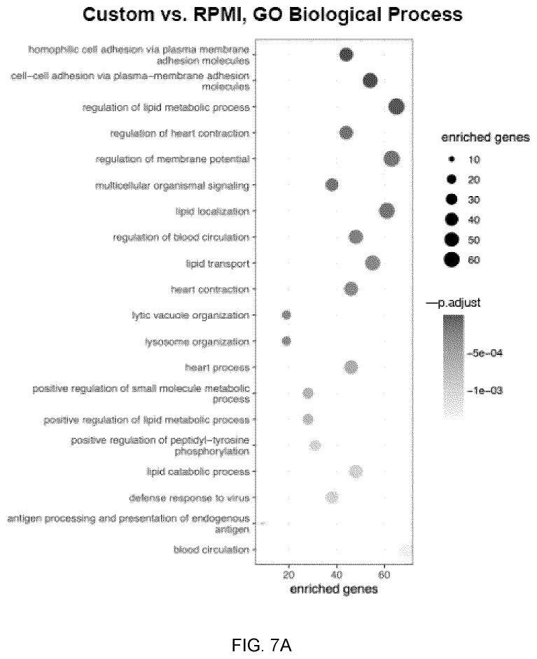

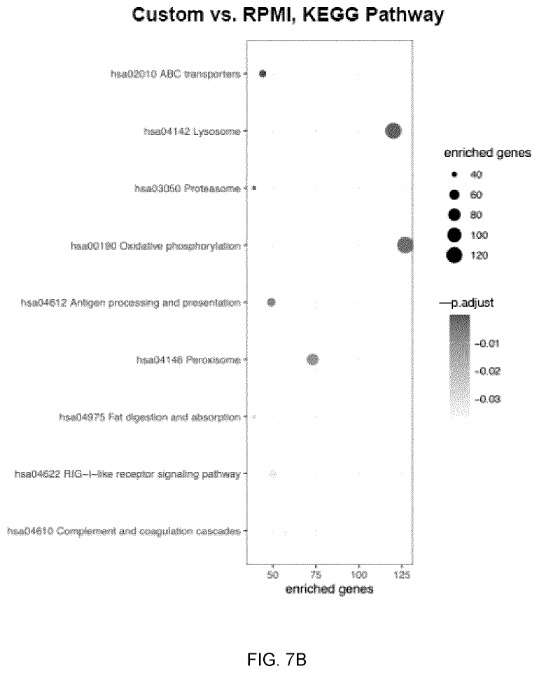

Gene set enrichment analysis (GSEA) was used to identify enriched GO biological process (i.e., functional) terms in iPSC-CMs treated with Custom or gold standard media. Relative to RPMI+B27, the top 20 terms enriched in Custom-treated cells were associated with general heart function, lipid metabolism and cell-cell adhesion (FIG. 7a). GSEA was also used to identify enriched KEGG pathways in iPSC-CMs treated with Custom or gold standard media (FIG. 7b). In agreement with respirometry data (above), oxidative phosphorylation-related genes were enriched in Custom-treated cells relative to the gold standard (FIG. 7b). Collectively, these results evidence that Custom medium enhances a number of general CM functions, particularly with respect to metabolism.

While transcriptomic analysis generally indicated that Custom medium promotes PSC-CM maturation relative to gold standards, several unexpected functional terms were enriched by GSEA. For instance, GO biological process terms associated with the IFN-y signalling pathway were among the top enriched terms in Custom-treated cells. Interestingly, several of the upregulated genes within these categories were associated with the response to DAMPs, which are generated by CMs during hypoxic stress [Ghigo A, Franco I, Morello F, Hirsch E (2014) Myocyte signalling in leucocyte recruitment to the heart. Cardiovasc Res 102:270-280]. While CMs generally only become hypoxic under pathological conditions in vivo, highly oxidative cell types may become hypoxic in static culture due to diffusion-limited reoxygenation of the cell-medium interface [Place T L, Domann F E, Case A J (2017) Limitations of oxygen delivery to cells in culture: An underappreciated problem in basic and translational research. Free Radic Biol Med 113:311-322.] Respirometry data, as well as KEGG pathway analyses indicated that Custom-treated cells are significantly more oxidative than existing gold standards; therefore, the enrichment of IFN-y-associated terms may be related to hypoxic stress.

In summary, the Custom medium formulation demonstrated more mature morphology than existing commercial gold-standard media and the homemade RPMI+B27 formulation. The Custom formulation exerted similar benefits on Ca2+ handling metrics, including both upstroke and relaxation kinetics, as well as a notably more pronounced response to the β-adrenergic agonist isoproterenol; Custom-treated cells also showed significantly higher relative sarcoplasmic Ca2+ capacity than STEMCELL medium when subjected to a bolus of caffeine. Together, these findings provided evidence of improved Ca2+ handling infrastructure within the cell, as well as a robustly-developed PKA pathway to provide differential β-adrenergic response. Custom medium-induced benefits to Ca2+ handling were seemingly matched by increases to oxidative metabolism, a hallmark of CM maturity. Custom-treated cells tended toward an increased per-cell oxygen consumption rate, which was also compounded upon mitochondrial uncoupling at a higher ratio than the gold-standard controls. Global proteomic analysis further supported these functional findings. Relative to the previously-mentioned gold standards, Custom-treated iPSC-CMs showed enriched expression of a wide range of proteins characteristic of mature CMs, including proteins associated with the cytoskeleton and contractility, action potential conduction, Ca2+ handling, protein trafficking, mitochondria, lipid trafficking and metabolism.

### Example 2. Inhibition of Spontaneous Beating and Microtissue Maturation

The presence of stable resting (diastolic) membrane potentials and the absence of spontaneous beating are hallmarks of mature working myocardium.

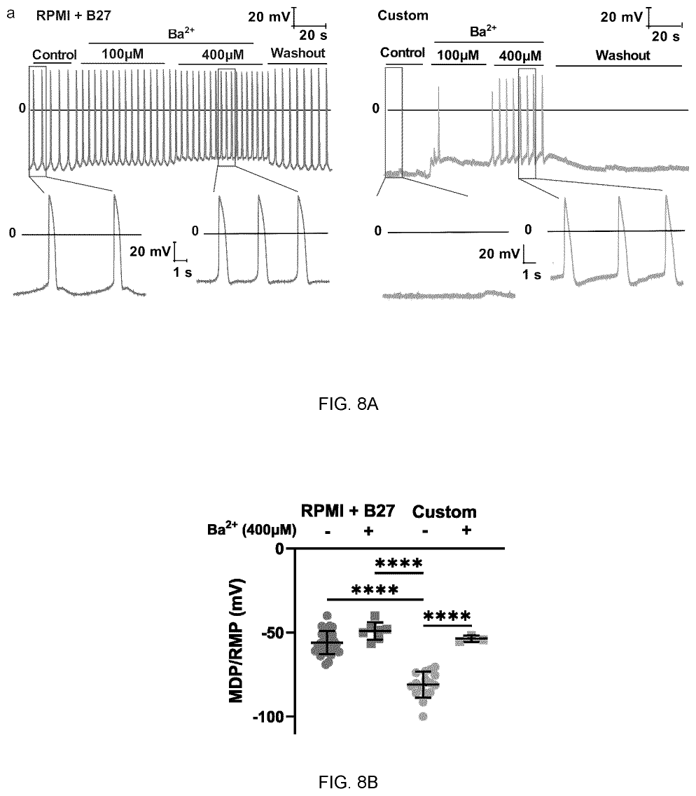

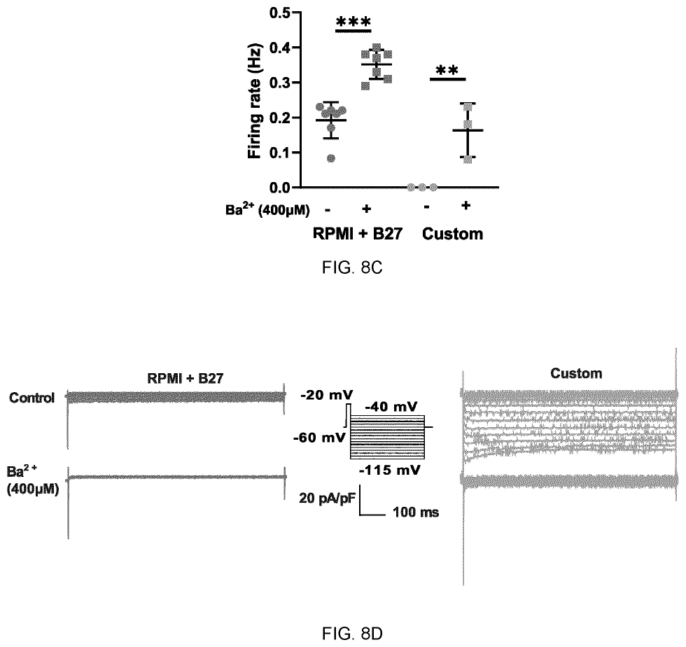

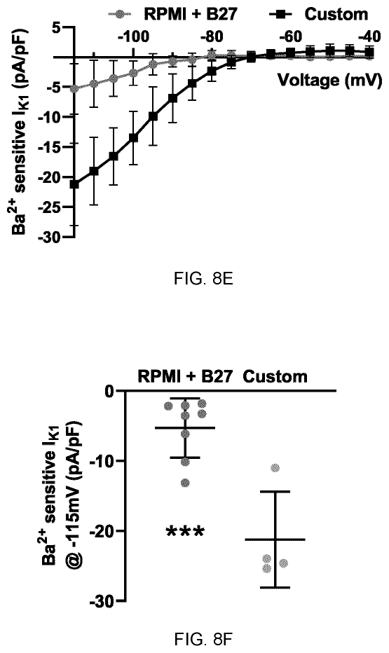

iPSC-CM monolayers (prepared as detailed in Example 1) treated with Custom medium were not spontaneously contractile, unlike RPMI+B27-treated cells, evidencing marked electrophysiological differences between treatments. To assess the electrical properties of cell cultures, intracellular voltages were measured in cells after 6 weeks of treatment with either Custom or RPMI+B27 (FIG. 8a). Action potentials (APs) were record in single iPSC-CMs or small clusters of iPSC-CMs (n=14 for Custom and n=30 for RPMI+B27) using sharp microelectrodes of resistances between 40 to 90 MO (filled with 3 mol L-1 KCl) with an Axopatch 200B amplifier. Intracellular voltages were digitized at a sample rate of 10 kHz (Digidata 1440 A plus pCLAMP 10.3, Molecular De-vices). Voltage recording was performed at room temperature and perfused with modified Tyrode's solution (pH 7.4) containing (in mmol L-1): NaCl (120), KCl (5) CaCl₂ (2), MgCl₂ (1), Na₂HPO₄ (0.84), MgSO₄ (0.28), KH₂PO₄ (0.22), NaHCO₃ (27), and glucose (5.6). These electrical differences were associated with classical differences in the resting diastolic membrane potentials. Specifically, RPMI+B27 cells underwent spontaneous de-polarization after reaching minimum diastolic membrane potentials (MDPs) of −56.1±6.8 mV (FIG. 8b), a pattern that is characteristic of immature cardiomyocytes, while Custom-treated cells displayed fixed resting diastolic membrane potentials (−81.1±7.7 mV). RPMI+B27-treated cell APs were accompanied simultaneously by contraction and Ca2+ transients (FIG. 8c) at a rate of ˜0.2 Hz. By contrast, Custom-treated cells did not display spontaneous action potentials (APs), beating or Ca2+ transients in the absence of electrical stimulation.

Voltage-clamp measurements were used to record whole-cell currents in single isolated iPSC-CMs using the patch-clamp technique at room temperature. The hardware was the same as for the AP recordings. The pipette solution (pH 7.2 adjusted with KOH) contained (in mmol L-1): K-gluconate (150), EGTA (5), HEPES (10), and MgATP (5). The series resistance of the pipettes was 3-4 MO. The external bath solution (pH 7.35-7.4 with NaOH) contained (in mmol L-1): NaCl (148), KCl (5.4), MgCl₂ (1.0), CaCl₂ (1.8), NaH₂PO₄ (0.4), glucose (5.5), and HEPES (15). The external perfusion solutions also contained nifedipine (5 μmol L-1) to block possible overlapping Ca2+ currents. IK1 was assessed by using voltage-steps from −115 mV to −20 mV (in 5 mV intervals) were applied for 500 ms from a membrane voltage of −60 mV. Currents were recorded at sampling rates of 10 kHz and analyzed using Clampfit 10.7 (Molecular Devices). Current-voltage (I-V) relationships were generated using peak currents as a function of membrane voltage. Currents were also recorded following the perfusion of BaCl₂ (100-400 μmol L-1), which is a potent and selective blocker of IK1 at such low concentrations [Duan, Q. et al. Spermine ameliorates ischemia/reperfusion injury in cardiomyocytes via regulation of autophagy. Am. J. Transl. Res. 8, 3976-3985 (2016).].

Previous studies have established that the absence of spontaneous APs in mature tissues is linked to the presence of background inward rectifier K+ channels (Ik1), which are able to “clamp” the resting membrane potential at values close to the equilibrium potential for K+ ions (EK) [Paci, M., Penttinen, K., Pekkanen-Mattila, M. & Koivumáki, J. T. Arrhythmia Mechanisms in Human Induced Pluripotent Stem Cell-Derived Cardiomyocytes. J. Cardiovasc. Pharmacol. 77, 300-316 (2020).]. To assess whether IK1 underlies the differences in spontaneous APs and beating between Custom and RPMI+B27, extracellular Ba2+ was applied at a dose (400 μmol L-1) that potentially blocks IK1 channels. While Ba2+ addition had minimal effect on either MDPs (49.2±5.3 mV) or the beating rates of RPMI+B27 cells, it caused Custom-treated cells to display spontaneous beating and APs accompanied by spontaneous depolarizations from MDPs of −53.7±1.8 mV, providing evidence that Custom-treated cells express high number of I_{K1} channels compared to RPMI+B27-treated cells.

In voltage-clamp studies, Custom-treated cells demonstrated robust background K+ currents with characteristic kinetic behaviour, classical inward rectification properties (FIG. 8d-e) and reversal properties (i.e., ˜EK), which are hallmark features of I_{K1}. Moreover, these background currents in the Custom treatment were eliminated by 400 μmol L⁻¹ Ba²⁺ (FIG. 8e). RPMI+B27 cells also displayed Ba²⁺-sensitive background K+ currents but these were ˜4-fold smaller than in Custom-treated cells (i.e., Ba²⁺-sensitive densities measured at −115 mV were −21.9±8.2 pA pF⁻¹ in Custom versus −5.5±3.9 pA pF⁻¹ in the RPMI+B27 treatment, FIG. 8f), consistent with the observed effects of Ba2+ on APs and beating of Custom- vs. RPMI+B27-treated cells.

It is notable that Custom-treated cells achieved I_{K1} densities roughly equivalent to those measured in isolated adult cardiomyocytes. Custom-treated iPSC-CMs were generally quiescent after approximately 3 weeks, and did not display APs or contractile spontaneity at 6 weeks unless I_{K1} was blocked by targeted Ba²⁺ application.

The Biowire II platform [Wang, E. Y. et al. Biowire Model of Interstitial and Focal Cardiac Fibrosis. ACS Cent. Sci. 5, 1146-1158 (2019)] was used to examine the impact of a maturation medium according to the present invention on parameters of CM function. These studies involved the creation of compacted multicellular muscle strips, followed by 3 weeks of culture in either the Custom medium modified to exclude 2-deoxyglucose (referenced in this Example as “Custom minus 2DG”) or RPMI+B27, and with or without continual electrical field pacing (1 Hz). 2-deoxyglucose was omitted for co-culture applications, due to toxicity concerns for primary fibroblasts at working concentrations.

Microtissues were created according to established methods (Wang, E. Y. et al. (2019). Briefly, iPSC-CMs mixed with primary human fibroblasts were cast in fibrin gels within embossed polystyrene molds and suspended between flexible poly(octamethylene maleate (anhydride) citrate) (POMaC) posts to allow for contraction against resistance. All strips were cultured in 10 cm cell culture dishes, containing 10 mL of medium changed twice weekly, with 0.1% penicillin-streptomycin.

Strips of up to 8 functional tissues were cultured 1 week in RPMI+B27 medium, to allow compaction, before assignments to treatments in either Custom minus 2DG or continued RPMI+B27 media, and with or without electrical field stimulation (10 V cm-1 at 1 cm, 1 Hz monophasic square wave pulses for 3 ms) via two graphite electrodes connected to an S48 physiological stimulator (Grass Technologies, USA).

Tissues were assessed for function at 0, 7, 14, and 21 d of treatment for baseline paced diastolic stress, peak systolic twitch stress, and stimulation force-frequency relationship up to 6 Hz using an IX71 microscope at 37° C. Polymeric wires were separately assessed for stiffness using a MicroSquisher (CellScale, Waterloo, Canada) to calculate absolute tissue diastolic and active forces. Twitch forces were calculated as the difference between diastolic and active forces. Stresses were derived from forces normalized to calculated cross-sectional tissue area (an ellipse of 5:3 aspect ratio at the tissue midpoint between polymeric wires based on existing cross-sectional imaging). Stress values were first analyzed using a 3-way repeated measures ANOVA (Medium×Stimulation×Time) to assess interactions between the effects of medium versus stimulation on systolic stress. If the 3-way interaction analyses failed to find significance, the were repeated by 2-way repeated measures ANOVA combined with a within-timepoints Tukey's HSD, as post-hoc test. Sphericity was not assumed and the Geisser-Greenhouse correction was applied when indicated.

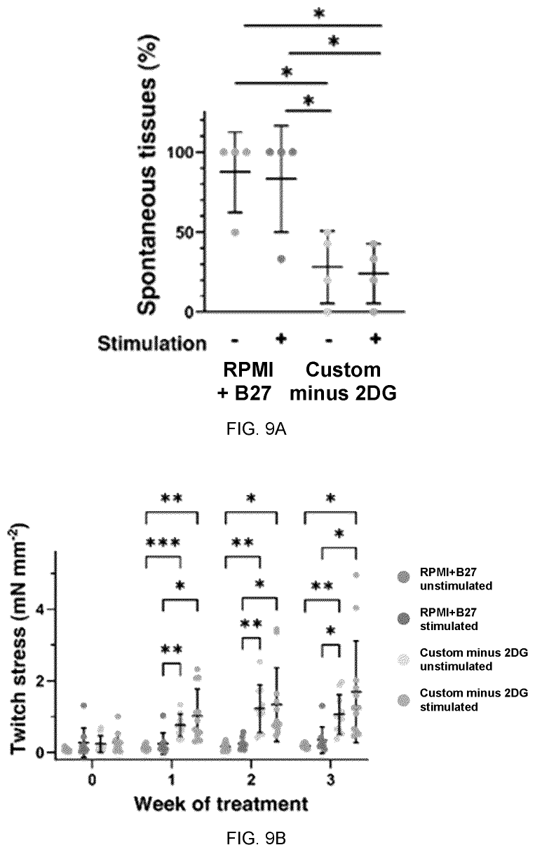

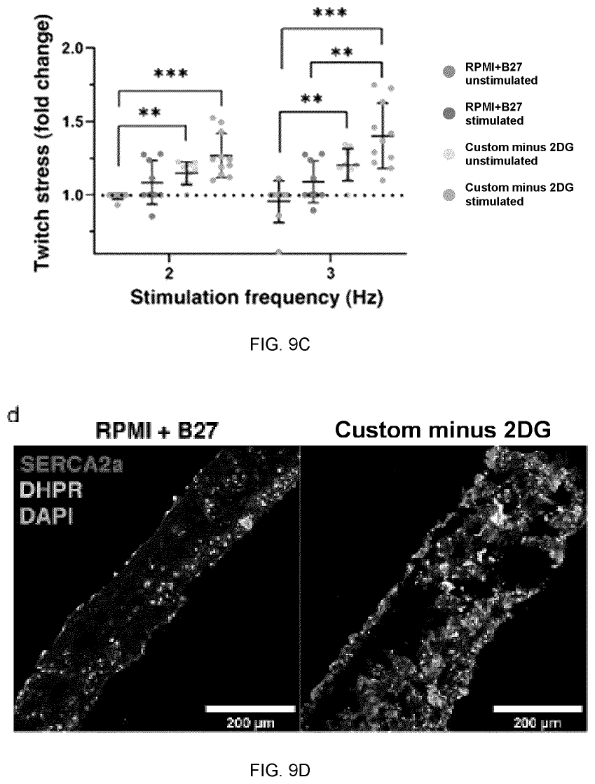

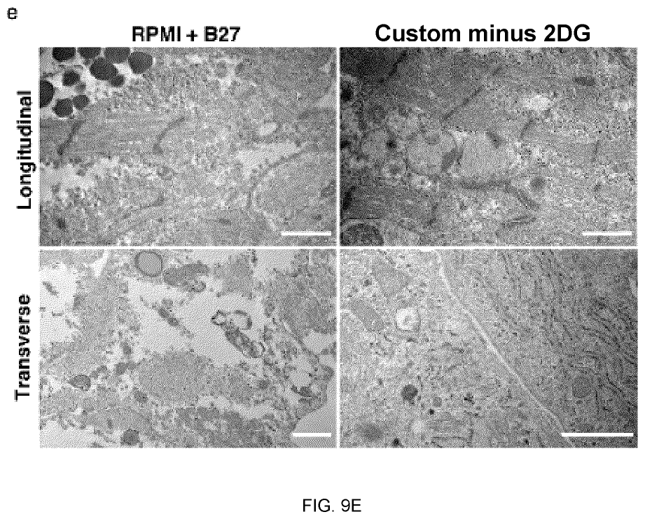

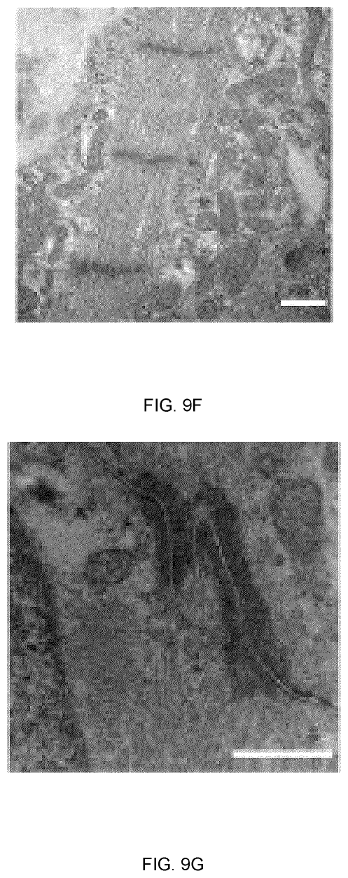

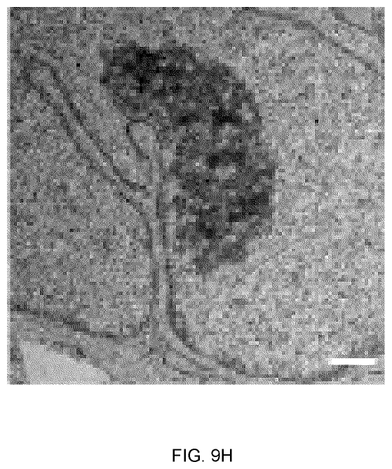

Biowire tissues in Custom minus 2DG media exhibited lower rates of spontaneous contractility than in RPMI+B27 (FIG. 9a) after 3 weeks in of treatment. Contractile forces normalized to cross-sectional area (i.e., twitch/systolic stress) was ˜5-fold higher with Custom minus 2DG versus RPMI+B27 treatment after 1, 2, or 3 weeks in culture (FIG. 9b), with or without electrical stimulation during the culture period. After 3 weeks in culture with electrical stimulation, the twitch stress was 1.69±1.42 mN mm-2 with Custom minus 2DG treatment vs. 0.35±0.37 mN mm-2 with RPMI+B27 treatment. By 3-way ANOVA, Custom minus 2DG treatment (p=0.0002) and culture time (p<0.0001) both independently enhanced twitch stress, and these parameters positively interacted (p<0.0001), establishing greater benefit of culture time in Custom minus 2DG medium versus the gold-standard control. Neither tissue morphology nor diastolic stresses were significantly affected by treatments. Furthermore, positive force-frequency responses after 3 weeks in culture were enhanced when measured at either 2 Hz or 3 Hz stimulation (relative to tissue-matched base-line stresses at 1 Hz) by both culture time (p<0.0001) and Custom minus 2DG treatment (p<0.0001) (FIG. 9c); again, there was a positive interaction (p<0.0001) between these factors.

Tissues treated for 3 weeks with the Custom minus 2DG formulation also exhibited higher-density SERCA2a and DHPR staining than those treated with RPMI+B27, similarly to in mono-layers (FIG. 9d). However, DHPR expression was variable between cells, suggesting stratification of phenotypes within the tissue. Compared to RPMI+B27 treatment, Custom minus 2DG-treated tissues under transmission electron microscopy demonstrated increased myofibrillar bundling, sarcomeric striation and zonation, mitochondrial density and myofibrillar association, electron-dense intercalated discs, and nuclear invaginations by the SR 37 (FIG. 9e-h).

What is claimed is:

1. An aqueous cell culture medium comprising.
a high-flux carbon source or central carbon metabolism inhibitor;
at least one secondary metabolite;
at least one of putrescine, leukemia inhibitory factor (LIF) and metformin.

2. The cell culture medium of claim 1 comprising about 0.001 mM to about 0.03 mM putrescine, about 0.1 ng/mL to about 10 ng/mL LIF and/or about 0.001 mM to about 0.25 mM metformin.

3. The cell culture medium of claim 1 or 2, wherein the high-flux carbon source or central carbon metabolism inhibitor is a lipid mixture, lactate, galactose or a combination of any of the foregoing.

4. The cell culture medium of claim 3, wherein the lipid mixture comprises cholesterol and one or more lipids selected from: linoleic acid, linolenic acid, myristic acid, oleic acid, palmitic acid, palmitoleic acid and stearic acid.

5. The cell culture medium of any one of claims 1 to 4, wherein the at least one secondary metabolite comprises creatinine, carnitine, taurine, ethanolamine or a combination of any of the foregoing.

6. The cell culture medium of claim 5, comprising about 0.001 mM to about 6.5 mM creatinine, about 0.001 mM to about 6.5 mM carnitine, about 0.001 mM to about 6.5 mM taurine, about 0.001 mM to 0.03 mM putrescine, and about 0.001 mM to about 0.01 mM ethanolamine.

7. The cell culture medium of any one of claims 1 to 6, further comprising one or more hormones selected from the group consisting of triiodothyronine (T3), insulin-like growth factor (IGF), hydrocortisone, human growth hormone, neuregulin and insulin, preferably T3, IGF, hydrocortisone, neuregulin and insulin.

8. The cell culture medium of claim 7, comprising about 0.001 ng/mL to 80 ng/mL T3, about 0.001 ng/mL to about 100 ng/mL IGF, about 0.001 ng/mL to about 80 ng/mL hydrocortisone, about 0.001 ng/mL to about 5 ng/mL neuregulin and about 0.75 gL to about 1.25 g/L insulin.

9. The cell culture medium of any one of claims 1 to 8, further comprising transferrin and selenium.

10. The cell culture medium of any one of claims 1 to 9, further comprising an antioxidant, preferably a synthetic antioxidant, preferably R-mercaptoethanol.

11. The cell culture medium of claim 1, comprising:

| | |
|---|---|
| 0 mM-0.03 mM | Putrescine |
| 0 mM-0.25 mM | Metformin |
| 0 ng/mL-10 ng/mL | Leukemia inhibitory factor |
| | | |
|---|---|---|
| 0-10 | mM | Lactate |
| 0-3 | g/L | A lipid mixture |
| | |
|---|---|
| 0 mM-40 mM | Galactose |
| 0 mM-6.5 mM | Creatine |
| 0 mM-6.5 mM | Carnitine |
| 0 mM-6.5 mM | Taurine |
| | | |
|---|---|---|
| 0-0.3 | mM | Ethanolamine |
| 0-1.25 | g/L | Insulin |
| 0-0.7 | g/L | Transferrin |
| 0-1 | mg/L | Selenium |
| 1-3 | wt % | Albumin |
| 0-0.1 | mM | β-mercaptoethanol |
| | |
|---|---|
| 0 ng/mL and 80 ng/mL | Triiodothyronine |
| 0 ng/mL and 100 ng/mL | Insulin-like growth factor |
| 0 ng/mL and 100 ng/mL | Human growth hormone |
| 0 ng/mL and 80 ng/mL | Hydrocortisone |
| 0 ng/mL and 5 ng/mL | Neuregulin |

12. The cell culture medium of any one of claims 1 to 11 comprising:

| | |
|---|---|
| 0.02 mM-0.025 mM | Putrescine |
| 0.05 mM-0.10 mM | Metformin |
| | | |
|---|---|---|
| 2-8 | ng/mL | Leukemia inhibitory factor |
| 0.1-3 | g/L | A lipid mixture |
| | |
|---|---|
| 15 mM-25 mM | Galactose |
| 0.1 mM-1 mM | Creatine |
| 0.1 mM-1 mM | Carnitine |
| 0.1 mM-1 mM | Taurine |
| 0.001 mM-0.01 mM | Ethanolamine |
| | | |
|---|---|---|
| 0.1-1.25 | g/L | Insulin |
| 0.1-0.7 | g/L | Transferrin |
| 0.1-1 | mg/L | Selenium |
| 1-3 | wt % | Albumin |
| | |
|---|---|
| 0.001 mM-0.1 mM | β-mercaptoethanol |
| 1 ng/mL-10 ng/mL | Triiodothyronine |
| 10 ng/mL-40 ng/mL | Insulin-like growth factor |
| 1 ng/mL-10 ng/mL | Hydrocortisone |
| .01 ng/mL-1 ng/mL | Neuregulin |

13. The cell culture medium of any one of claims 1 to 12, wherein the high-flux carbon source comprises 0.001 mM to 20 mM 2-deoxyglucose, preferably 5 mM to 10 mM 2-deoxyglucose.

14. The cell culture medium of any one of claims 1 to 12, wherein the medium is substantially free of 2-deoxyglucose.

15. The cell culture medium according to any one of claims 1 to 14, wherein the medium is a serum-free medium.

16. The cell culture medium of any one of claims 1 to 15, wherein the medium further comprises Media 199.

17. The cell culture medium of any one of claims 1 to 16 further comprising at least one antibiotic, preferably penicillin and/or streptomycin.

18. A kit comprising the culture medium according to any one of claims 1 to 17 and immature cardiomyocytes or undifferentiated pluripotent embryonic stem cells (PESC) and/or induced pluripotent stem cells (iPSC) and a differentiation medium for differentiating PESC and/or iPSC into cardiomyocytes.

19. A composition for improving the cardiomyocyte maturing properties of a cell culture medium, the composition comprising one or more of putrescine, leukemia inhibitory factor (LIF) and metformin.

20. The composition according to claim 19 comprising any two of putrescine, LIF and metformin or all of putrescine, LIF and metformin.

21. The composition of claim 19 or 20, wherein the composition is a solid or semi-solid, and wherein the composition can be dissolved in an aqueous solution so as to obtain the cell culture medium of any one of claims 1 to 17.

22. A method of maturing immature cardiomyocytes to obtain adult-like cardiomyocytes comprising:
contacting immature cardiomyocytes with a culture medium according to any one of claims 1 to 17 for a time sufficient to effect maturation of the immature cardiomyocytes.

23. The method of claim 22, further comprising differentiating PESC and/or iPSC to obtain the immature cardiomyocytes.

24. The method of claim 22 or 23, wherein the immature cardiomyocytes are contacted with the culture medium for a period of 10-45 days.

25. The method of claim 24, wherein the immature cardiomyocytes are contacted with the culture medium for a period of 14-21 days.

26. The method of any one of claims 22 to 25, wherein the cells are cultured at a concentration of 50K to 250K cells per cm².

27. The method of any one of claims 22 to 26, comprising co-culturing the immature cardiomyocytes with non-cardiomyocyte cells, preferably epicardial cells and/or cardiac fibroblasts.

28. The method of any one of claims 22 to 27 comprising contacting the immature cardiomyocytes with the culture medium in a 3D tissue culture system, preferably an engineered tissue or organoid.

29. Adult-like cardiomyocytes matured by a method of any one of claims 22 to 28.

30. A composition comprising the adult-like cardiomyocytes of claim 29 and a pharmaceutically acceptable carrier or biocompatible scaffold.
